(19) United States
(12) Patent Application Publication
(10) Pub. No.: US 2010/0215603 A1
(43) Pub. Date: Aug. 26, 2010

(54) Body Surface Protecting Composition

(76) Inventors: Kentarou Kanda, Tokyo (JP); Yokio Yamawaki, Tokyo (JP); Yamato Saitou, Tokyo (JP)

Correspondence Address: BIRCH STEWART KOLASCH & BIRCH, PO BOX 747, FALLS CHURCH, VA, 22040-0747, US

(21) Appl. No.: 11/922,101

(22) PCT Filed: Jun. 13, 2006
(86) PCT No.: PCT/JP2006/311789

§ 371 Date: Dec. 13, 2007

(30) Foreign Application Priority Data
Jun. 14, 2005 (JP) 2005-173233
Aug. 16, 2005 (JP) 2005-235803

(51) Int. Cl. A61K 31/197; C07C 233/48; A61K 8/92; A61Q 17/00; A61Q 5/00; A61P 17/00; A61K 31/56; A61K 31/575; A61P 29/00

(52) U.S. Cl. 424/70.1; 554/57; 514/563; 514/171

(57) ABSTRACT

A body surface protecting composition, comprising at least one type of multi-chain multiple hydrophilic group-type compound having two or more hydrophobic groups and two or more hydrophilic groups in the molecule, which has an excellent effect of accelerating the recovery of the barrier function of skin and hair and preventing degradation of that function.

### TECHNICAL FIELD

The present invention relates to a composition that is used in applications to prevent or reduce disorders on a body surface (skin, hair, and the like), specifically relates to a body surface protecting composition. In particular, the present invention relates to a body surface protecting composition which has the excellent effects of accelerating the recovery of the barrier functions of skin and hair, and preventing degradation of those functions.

### BACKGROUND ART

Skin barrier functions include a function that prevent invasion by foreign matter from outside the skin into a body, and a function that prevents water content in the body from escaping to the outside. When the barrier function of the skin is degraded, rough skin or inflammation of the skin such as rash can result from external stimulation, and dry chapped skin can result from an increase in the transpiration of water from the skin, and therefore itchiness and skin trouble can easily occur. Furthermore, the barrier function of the hair is primarily a function of preventing water and aqueous components from escaping to the outside. When the barrier function of the hair is reduced, the tensile strength and viscoelasticity of the hair will be degraded, the feel on the skin will be poor, hair styling will be difficult, and not only will this induce a deterioration in appearance such as luster, but will also cause damage such as split ends and broken hair.

Conventionally moisturizing agents have been added to cosmetic materials in order to improve and prevent this type of dry condition in skin and hair. For example, patent document 1 discloses a cosmetic material for skin that contains a polyol such as glycerin or ethylene glycol, or urea, hyaluronic acid or an amino acid or the like as a moisturizing agent. However, with these cosmetic materials, the effect is temporary and is insufficient for resolving the aforementioned problems. Furthermore, this cosmetic material is not used for applications to reduce and prevent damage to the surface of the body, and there is no comment whatsoever concerning an effect of accelerating improvements in the barrier function of the skin or preventing a degradation in that function. Furthermore, according to recent dermatological research, simple application of a moisturizing agent onto the surface of skin will increase the moisture of the skin for a short period of time, but in actuality may hinder the intrinsic function of the skin to retain moisture in the skin, and is known to damage the condition of the skin.

Furthermore, patent document 2 for instance discloses a composition comprising an anionic surfactant with two chains and two hydrophilic groups, and discloses the use as a cosmetic material with moisturizing effects. However, this composition is also not used for applications to reduce and prevent damage to the surface of the body, and it dose not disclose any effect of accelerating improvements in the barrier function of the skin or preventing any degradation in that function, and does not disclose a compound that has those effects.

Furthermore, patent document 3 discloses a moisture retaining composition which does not feel sticky by the combined use of a double chain amide compound with a polyhydric alcohol. However, this moisture retaining material is essentially a polyhydric alcohol, and similar to patent document 1, is not used for applications to reduce and prevent damage to the surface of the body, and it does not disclose any effect of accelerating improvements in the barrier function of the skin or preventing a degradation in net function, and only discloses the fact that a double chain amide compound works to reduce the stickiness when the composition is applied.

Patent document 4 discloses a composition with low irritability that contains an anionic surfactant with a double chain bipolar acyl group, but it only disclose the low irritability of the composition, and dose not disclose any application for preventing or reducing damage to the surface of the body, nor any effect of proactively accelerating an improvement of the skin barrier function or preventing a degradation of that function.

Patent document 5 discloses an agent to improve skin damage and metabolic disorders using a Gemini type surfactant, but the Gemini surfactant disclosed in this document is only a carrier for transporting physiologically active materials through skin or membrane, and does not show an effect of accelerating an improvement in the skin barrier function or preventing deterioration of that function.

On the other hand, a type of sphingolipid such as ceramides obtained either naturally or synthetically are well-known as substances which improve the skin barrier function, and for example, patent document 6 discloses a liquid crystal composition using various types of ceramides, patent document 7 discloses an external medicine that contains ceramides or substances with a structure similar to a ceramide, and patent document 8 discloses a skin care agent that contains ceramides, and discloses a composition that improves skin turn over, improves skin roughness, and has excellent permeability and moisture retaining properties. However, all of the ceramides used in these inventions have low water solubility, and not only cause difficulty when creating a composition, but also require improvisations in order to permeate into the skin and hair, and cause restrictions on the degree of freedom of the formulation.

Therefore there is demand for a raw material which has a strong effect at accelerating the recovery of the barrier function of skin and hair or in other words the surface of the body, and has good water solubility.

On the other hand, in patent document 9, the present inventors have disclosed a surfactant consisting of a composition containing an acyl group comprising an N-long chain acyl acidic amino acid derivative made by reacting N-long chain acyl acidic amino acid anhydride with at least one compound selected from compounds which have m functional groups of at least one type selected from a group consisting of a hydroxyl group, an amino group, and a thiol group in the molecule, as well as a manufacturing method thereof. In this publication, the surfactant obtained provides surface activity at low concentrations and has been confirmed to have low irritability.

Patent document 1 Japanese Patent Application Laid-open No. 2000-191499
Patent document 2 Japanese Patent Application Laid-open No. H10-218754
Patent document 3 Japanese Patent Application Laid-open No. H10-203956
Patent document 4 Japanese Patent Application Laid-open No. H10-219278
Patent document 5 WO2005/39642
Patent document 6 Japanese Patent Application Laid-open No. H9-124432
Patent document 7 Japanese Patent Application Laid-open No. H5-213731
Patent document 8 Japanese Patent Application Laid-open No. 2001-39859
Patent document 9 Japanese Patent Application Laid-open No. 2002-167313

### DISCLOSURE OF INVENTION

### Problems to be Solved by the Invention

An object of the present invention is to provide a body surface protecting composition which has an excellent effect of accelerating recovery of the barrier function of skin and hair when that function has deteriorated due to skin trouble or the like.

As a result of diligent investigations, the present inventors have discovered that a multi-chains and multiple hydrophilic group-type compound that has two or more hydrophobic groups and two or more hydrophilic groups in a molecule has an excellent effect of improving the barrier function of the skin and hair that is required because of skin trouble or the like.

As described above, this multi-chain multiple hydrophilic group-type compound is known to be a surfactant, but the effect of improving the barrier function of skin and hair is entirely unknown.

Furthermore, single chain single hydrophilic group-type compounds which have essentially the same basic constitutional units as multi-chain multiple hydrophilic group-type compounds can not provide the effect of accelerating recovery of the barrier function of skin, and conversely cause the barrier function to deteriorate. Therefore, it was unexpected that multi-chain multiple hydrophilic group-type compounds have the effect of improving the barrier function of skin and hair.

Furthermore, the present inventors have discovered that the effect of improving the barrier function of skin and hair with the present invention is increased when the multi-chain multiple hydrophilic group-type compound forms a liquid crystal, and the liquid crystal formation is facilitated by combining a oil-based component with the multi-chain multiple hydrophilic group-type compound.

In other words, the present invention is a body surface protecting composition comprising, (A) at least one type of multi-chain multiple hydrophilic group-type compound having two or more hydrophobic groups and two or more hydrophilic groups in the molecule.

In the multi-chain multiple hydrophilic group compound of (A), it is preferable that at least one of the hydrophobic groups is an acyl group, and at least one of the hydrophilic groups is a carboxyl group, sulfonate group, sulfate ester group, phosphate ester group, or a salt thereof, and it is more preferable that the aforementioned multi-chain multiple hydrophilic group-type compound further has an amino acid residue in the molecule.

Furthermore, a preferred multi-chain multiple hydrophilic group-type compound of (A) is a compound expressed by the following formula (1).

[C1]

Qn-X (1)

(In formula (1), X represents a spacer with a molecular weight of one million or less, has m functional group residues that may be the same or different, and may have other functional groups. Q represents a functional group as expressed by the following formula (2), the functional groups may be the same or different.

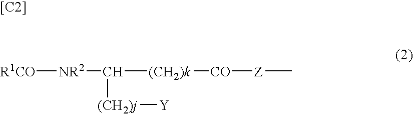

In formula (2), Z represents a site of bonding with a functional group residue contained in X, R¹ represents a hydrocarbon group that may be substituted with a saturated or unsaturated substitution group of from 2 to 20 carbon atoms, R² represents a hydrogen atom or a lower alkyl group of from 1 to 3 carbon atoms which may also be substituted with a substitution group, and Y represents a carboxyl group, a sulfonate group, a sulfate ester group, a phosphate ester group, or a salt thereof. j and k independently represent either 0, 1, or 2, but j and k cannot both be 0 at the same time. n is an integer from 2 to 20. Furthermore, m is an integer such that m≧n.).

In particular, the aforementioned multi-chain multiple hydrophilic group-type compound of (A) preferably is a compound expressed by the following formula (3).

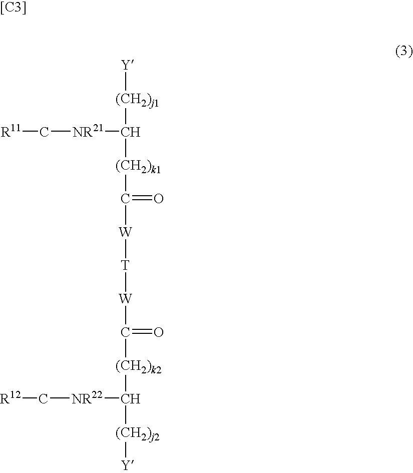

(In formula (3), R¹¹ and R¹² each independently represent a hydrocarbon group which may be substituted with a saturated or unsaturated substitution group of from 8 to 20 carbon atoms, R²¹ and R²² each independently represent either a hydrogen atom or a lower alkyl group of from 1 to 3 carbon atoms that may be substituted with a substitution group, each Y′ independently represents a carboxyl group or a salt thereof, each W independently represents —O—, —S—, or —NR′— (where R′ represents a hydrogen atom or a hydrocarbon group of from 1 to 10 carbon atoms which may be substituted with a substitution group), and T represents a spacer of from 1 to 20 carbon atoms. j¹, j², k¹, and k² independently represent either 0, 1, or 2, but j¹ and k¹, and j² and k² cannot be both 0 at the same time.).

Furthermore, the multi-chain multiple hydrophilic group-type compound of (A) preferably forms a liquid crystal, and therefore the body surface protecting composition also preferably comprise an oil component (B).

In the present invention, the multi-chain multiple hydrophilic group-type compound refers to a compound that has two or more hydrophobic groups and two or more hydrophilic groups in a molecule, and the hydrophobic groups are not necessarily restricted to chain compounds.

### EFFECT OF THE INVENTION

The composition of the present invention has an excellent effect of accelerating recovery of the barrier function of skin and hair when that function has deteriorated due to skin trouble or the like. Furthermore, the composition of the present invention also has an excellent effect of restoring damaged tips of hair.

### BEST MODE FOR CARRYING OUT THE INVENTION

The present invention will be described below in detail with a particular focus on working examples. (A) that is used in the body surface protecting composition of the present invention is a multi-chain multiple hydrophilic group-type compound which has two or more hydrophobic groups and two or more hydrophilic groups in a molecule, and may be any commonly known compound that has this structure. Furthermore, the two or more hydrophobic groups and hydrophilic groups can be the same or different.

The hydrophilic group of the multi-chain multiple hydrophilic group-type compound of (A) can be a carboxyl group, sulfonate group, sulfate residue, phosphate residue or a salt thereof, oxyalkylene group, polyethylene glycol group, amino group, quaternary ammonium group, pyridinium group, sulfonium group, as well as a salt thereof.

The hydrophobic group of the multi-chain multiple hydrophilic group-type compound of (A) can be a saturated or unsaturated straight chain, branched chain, or cyclical chain group of from 2 to 20 carbon.

Specific examples of the hydrophobic group of the multi-chain multiple hydrophilic group-type compound of (A) include n-acetyl, n-propyl, n-butyl, n-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, and n-eicosyl and the like, as well as branched chain isomers thereof and corresponding unsaturated residues with 1, 2, or 3 unsaturated-bonds. Of these hydrophobic groups, long chain hydrocarbon groups of from 8 to 20 carbon atoms are preferable.

The hydrophobic group of the multi-chain multiple hydrophilic group-type compound of (A) can be an acyl group derived from a saturated or unsaturated aliphatic acid. Of these, hydrophobic groups derived from saturated or unsaturated aliphatic acids of from 2 to 20 carbon atoms are preferable and those of from 8 to 20 carbon atoms are even more preferable. Their form may be straight chain, branched chain, or cyclic.

Examples of these acyl groups include acyl groups derived from straight chain aliphatic acids such as acetic acid, propionic acid, butyric acid, pentanoic acid, hexanoic acid, hexanoic acid, caprylic acid, pelargonic acid, caprionic acid, undecanoic acid, lauric acid, tridecanoic acid, myristic acid, pentadecanoic acid, palmitic acid, margarinic acid, stearic acid, nonadecanoic acid, and alginic acid; branched chain aliphatic acids such as 2-butyl-5-methyl pentanoic acid, 2-isobutyl-5-methyl pentanoic acid, dimethyl octanoic acid, dimethyl nonanoic acid, 2-butyl-5-methyl hexanoic acid, methyl undecanoic acid, dimethyl decanoic acid, 2-ethyl-3-methyl nonanoic acid, 2,2-dimethyl-4-ethyl octanoic acid, methyl docosanoic acid, 2-propyl-3-methyl nonanoic acid, methyl tridecanoic acid, dimethyl dodecanoic acid, 2-butyl-3-methyl nonanoic acid, methyl tetradecanoic acid, ethyl tridecanoic acid, propyl dodecanoic acid, butyl undecanoic acid, pentyl decanoic acid, hexyl nonanoic acid, 2-(3-methylbutyl)-3-methyl nonanoic acid, 2-(2-methylbutyl)-3-methyl nonanoic acid, butylethyl nonanoic acid, methyl pentadecanoic acid, ethyl tetradecanoic acid, propyl tridecanoic acid, butyl dodecanoic acid, pentyl undecanoic acid, hexyldecanoic acid, heptylnonanoic acid, dimethyltetradecanoic acid, butylpentyl heptanoic acid, trimethyl tridecanoic acid, methyl hexadecanoic acid, ethyl pentadecanoic acid, propyl tetradecanoic acid, butyl tridecanoic acid, pentyl dodecanoic acid, hexyl undecanoic acid, heptyl decanoic acid, methylheptyl nonanoic acid, dipentyl heptanoic acid, methyl heptadecanoic acid, ethyl hexadecanoic acid, ethyl hexadecanoic acid, propyl pentadecanoic acid, butyl tetradecanoic acid, pentyl tridecanoic acid, hexyl dodecanoic acid, heptyl undecanoic acid, octyl decanoic acid, dimethyl hexadecanoic acid, methyloctyl nonanoic acid, methyl octadecanoic acid, ethyl heptadecanoic acid, dimethyl heptadecanoic acid, methyloctyl decanoic acid, methylnon adecanoic acid, methyl nonadecanoic acid, dimethyl octadecanoic acid, butyl heptylnonanoic acid, and the like; straight chain monoene acids such as octenoic acid, nonenoic acid, decenoic acid, caproleic acid, undecylenic acid, lindelic acid, obtusilic acid, laurolenic acid, tridecenic acid, tsuzuic acid, myristolenic acid, pentadecenic acid, hexadecenic acid, palmitolenic acid, heptadecenic acid, octadecenic acid, oleic acid, nonadecenic acid, and gondoic acid; branched monoene acids such as methyl heptenoic, methyl nonenoic acid, methyl undecenoic acid, dimethyldecenoic acid, methyldodecenoic acid, methyltridecenoic acid, dimethyldodecenoic acid, dimethyltridecenoic acid, methyloctadecenoic acid, dimethylheptadecenoic acid, and ethyloctadecenoic acid; diene or triene acids such as linoleic acid, linoelaidic acid, eleostearic acid, linolenic acid, linolenelaidic acid, pseudoeleostearic acid, vanillinic acid, and arachidonic acid; acetylenic acids such as octynoic acid, nonynoic acid, decynoic acid, undecynoic acid, dodecynoic acid, tridecynoic acid, tetradecynoic acid, pentadecynoic acid, heptadecynoic acid, octadecynoic acid, nonadecynoic acid, and dimethyl octadecyne; cyclic acid such as methylene octadecenoic acid, methylene octdecanoic acid, aleprolic acid, aleprestic acid, aleprylic acid, alepric acid, hydnocarpic acid, chaulmoogric acid, gorlic acid, α-cyclopentyl acid, α-cyclohexyl acid, and α-cyclopentylethyl acid.

An acyl group derived from an aliphatic acid obtained from a natural oil is also preferably used, and acyl groups derived from mixture of aliphatic acids obtained from natural oils that include 80% or more of saturated or unsaturated fatty acids of from 2 to 20 carbon atoms are preferable. Examples of acyl groups derived from aliphatic acid obtained from natural oils include acyl groups and the like derived from coconut oil fatty acid, palm oil fatty acid, linseed oil fatty acid, sunflower oil fatty acid, soybean oil fatty acid, sesame oil fatty acid, castor oil fatty acid, olive oil fatty acid, camellia oil fatty acid, vegetable oil fatty acids, palm kernel oil fatty acid and the like.

These acyl groups can be used in combinations of 2 or more.

When the multi-chain multiple hydrophilic group-type compound of (A) is a salt, examples can include alkali metal salts, alkali earth metal salts, polyvalent metal salts, ammonium salts, organic amine salts, and basic amino acid salts and the like. Specific examples of these salts include one or more types of salt arbitrarily selected from salt of alkali metals such as sodium, potassium, and lithium, alkali earth metals such as calcium and magnesium, metals such as aluminum, zinc, iron, cobalt, titanium, and zirconium, organic amines such as ammonia, monoethanolamine, diethanolamine, triethanolamine, and triisopropanolamine, and basic amino acids such as arginine and lysine. Of these salts, sodium salt, potassium salt, organic amine salt, and basic amino acid salt are particularly preferable.

In the present invention, at least one type of the multi-chain multiple hydrophilic group-type compound of (A) is preferably a compound expressed by the following formula (1).

[C 4]

Qn-X (1)

The compound expressed by formula (1) can take a several optical isomer structure such as a D-isomer, an L-isomer, or a racemic body, and either type can be used.

In formula (1), X represents a spacer with a molecular weight of one million or less, has m functional group residues that may be the same or different, and may have other functional groups, and the form can be straight chain, branched chain, or cyclical chain (aromatic hydrocarbon chain). A spacer as used in the present invention refers to a site that connects a plurality of atom groups in a compound.

The aforementioned functional group residue can be a residue of a hydroxyl group, amino group, or a thiol group, or in other words —O—, —S—, or —NR′— (where R′ represents a hydrogen atom or a hydrocarbon group of from 1 to 10 carbon atoms and which may also be substituted with a substitution group).

Specific examples of X include amino acids such as serine, threonine, cysteine, cystine, cystine disulfoxide, cystathionine, methionine, arginine, lysine, tyrosine, histidine, tryptophan, and oxyproline; compounds with an amino group and a hydroxyl group in a molecule, such as aminoethanol, aminopropanol, aminobutanol, aminopentanol, aminohexanol, aminopropanediol, aminoethylethanolamine, aminoethylaminoethanol, aminoethylaminoethanol, aminocreosol, aminonaphthol, aminonaphthol sulfonic acid, aminohydroxybenzoic acid, aminohydroxybutanoic acid, aminophenol, aminophehethyl alcohol, and glucosamine; compounds with a thiol group and a hydroxyl group in a molecule, such as mercaptoethanol, mercaptophenol, mercaptopropanediol, and glucothiose; and compounds with a thiol group and an amino group in a molecule, such as aminothiophenol andaminotriazolethiol. X may be a protein or a peptide or the like, as well as a residue of a hydrolyzed product thereof.

Specific examples of X include dihydroxyl compounds such as ethylene glycol, 1,2-propanediol, 1,3-propanediol, 1,2-butanediol, 1,3-butanediol, 1,4-butanediol, pentanediol, 1,6-hexanediol, cyclohexanediol, dimethylol cyclohexane, neopentyl glycol, 1,8-octanediol, 2,2,4-trimethyl-1,3-pentanediol, isoprene glycol, 3-methyl-1,5-pentanediol, sorbite, catechol, resorcin, hydroquinone, bis phenol A, bis phenol F, hydrated bis phenol A, hydrated bis phenol F, dimerdiol, dimethylolpropionic acid, dimethylolbutanoic acid, tartaric acid, dihydroxytartaric acid, mavalonic acid, 3,4-dihydroxycinnamic acid, 3,4-dihydroxyhydrocinnamic acid, hydroxybenzoic acid, dihydroxystearic acid, dihydroxyphenylalanine, as well as isomers thereof; trihydric polyhydroxyl compounds such as glycerin, trioxyisobutane, 1,2,3-butanetriol, 1,2,3-pentanetriol, 2-methyl-1,2,3-propanetriol, 2-methyl-2,3,4-butanetriol, 2-ethyl-1,2,3-butanetriol, 2,3,4-pentanetriol, 2,3,4-hexanetriol, 4-propyl-3,4,5-heptanetriol, 2,4-dimethyl-2,3,4-pentanetriol, 1,2,4-butanetriol, 1,2,4-pentanetriol, trimethylolethane, trimethylolpropane, diethanolamine, triethanolamine, and trihydroxystearic acid; quadrahydric polyhydroxyl compounds such as pentaerythritol, erythritol, 1,2,3,4-pentanetetrol; 2,3,4,5-hexanetetrol, 1,2,4,5-pentanetetrol, 1,3,4,5-hexanetetrol, diglycerin, and sorbitan; pentahydric poly hydroxyl compounds such as adonitol, arabitol, xylytol, and triglycerine; hexahydric polyhydroxyl compounds such as dipentaerythritol, sorbitol, mannitol, iditol, inositol, dulcitol, talose, and allose; as well as condensate products thereof, and polyglycerin and the like.

Examples of sugars include residues of polyhydroxyl compounds such as monosaccharides such as tetroses such as erythrose, threose, and erythrulose; pentoses such as ribose, arabinose, xylose, licsose, xylulose, and rubulose; and hexoses such as allose, artrose, glucose, mannose, gulose, idose, galactose, talose, fructose, sorbose, psicose, and tagatose; and oligosaccharides such as maltose, isomaltose, cellobiose, genthiobiose, melibiose, lactose, turanose, trehalose, saccharose, mannitolylose, cellotriose, genthianose, raffinose, melezitose, cellotetrose, and stachyose, and the like.

X may also be residues of other sugars such as heptose, deoxy sugar, amino sugar, thio sugar, sereno sugar, aldonic acid, uronic acid, saccharic acid, ketoaldonic acid, anhydro sugar, unsaturated sugar, sugar ester, sugar ether, and glycoside, as well as polysaccharides such as starch, glycogen, cellulose, chitin, and chitosan, as well as hydrolyzed residues thereof.

Specific examples of X also includes residues of polyamino compounds such as aliphatic diamines such as N,N′-dimethylhydrazine, ethylenediamine, N,N′-dimethylethylenediamine, diaminopropane, diaminobutane, diaminopentane, diaminohexane, diaminoheptane, diaminooctane, diaminononane, diaminodecane, diaminododecane, diaminoadipic acid, diaminopropanoic acid, diaminobutanoic acid and various isomers thereof; aliphatic triamines such as diethylenetriamine, triaminohexane, triaminododecane, 1,8-diamino-4-aminomethyl-octane, 2,6-diaminocapric acid-2-aminoethyl ester, 1,3,6-triaminohexane, 1,6,11-triamino undecane, di(aminoethyl) amine, as well as various isomers thereof; alicyclic polyamines such as diaminocyclobutane, diaminocyclohexane, 3-aminomethyl-3,5,5-trimethylcyclohexylamine, and triaminocyclohexane; aromatic polyamines such as diaminobenzene, diaminotoluene, diaminobenzoic acid, diaminoanthraquinone, diaminobenzenesulfonic acid, diaminobenzoic acid, and various isomers thereof; araliphatic polyamine such as diaminoxylene, di(aminomethyl)benzene, di(aminomethyl)pyridine, di(aminomethyl)naphthalene, and various isomers thereof; hydroxyl group substituted polyamines such as diaminohydroxypropane and various isomers thereof, and the like.

Furthermore, specific examples of X also include residues of dithiol compounds such as dithioethylene glycol, dithioerythritol, and dithiothreitol.

X preferably has from 1 to carbons, more preferably from 1 to 20 carbons. Furthermore, X is preferably in a naturally existing form, because the biodegradability will be excellent.

When X is an amino acid residue, the affinity of the compound expressed in formula (1) towards skin and hair will be high, and the compound expressed in formula (1) will easily permeate into skin and hair so the effect of improving the barrier function will be further accelerated.

In formula (1), Q is a substitution group expressed in formula (2), and each functional groups may be the same or different.

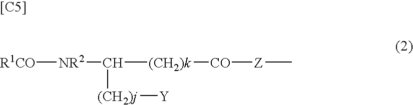

In formula (2), R¹ represents a hydrocarbon group that may be substituted with a saturated or unsaturated substitution group of from 2 to 20 carbon atoms, and may be a straight chain, branched chain, or ring. A saturated or unsaturated hydrocarbon group of from 8 to 20 carbon atoms is preferable.

In formula (2), R² represents a hydrogen atom or a lower alkyl group of from 1 to 3 carbon atoms such as a carboxyl group, a sulfonate group, a sulfate ester group, a phosphate ester group, or salts thereof, and may also be substituted. Examples include a methyl group, an ethyl group, a propyl group, an isopropyl group, a hydroxymethyl group, a hydroxyethyl group, a hydroxy(iso)propyl group, a dihydroxy(iso)propyl group, a carboxymethyl group, a carboxyethyl group, a carboxypropyl group, and a sulfoethyl group.

In formula (2), Y represents a carboxyl group, a sulfonate group, a sulfate ester group, a phosphate ester group, or a salt thereof. Salts of carboxyl groups, sulfonate groups, sulfate ester groups, and phosphate ester groups include alkali metal salts, alkali earth salts, polyvalent metal salts, ammonium salts, organic amine salts, and basic amino acid salts, and specific examples include alkali metals such as sodium, potassium, and lithium; alkali earth metals such as calcium and magnesium; metals such as aluminum, zinc, iron, cobalt, titanium, and zirconium; organic amines such as ammonia, monoethanolamine, diethanolamine, triethanolamine, and triisopropanolamine, and basic amino acids such as arginine and lysine.

In formula (2), Z is a site of a bonding with a functional group residue contained in X.

A preferred substitution group Q expressed in formula (2) is a residue of an N-acylated acidic amino acid because the effect of improving the barrier function of the skin and hair will be further enhanced. Acidic amino acid as used in the present invention refers to a monoaminodicarboxylic acid having 2 carboxyl groups and 1 amino group present in a molecule, and the amino group may be an N-methyl group or an N-ethyl group. The substitution group Q expressed in formula (2) can be any optical isomers of a D-isomer, L-isomer or racemic body, but Q is preferably a residue of an L-acidic amino acid compound because the biodegradability will be excellent.

Examples of acidic amino acids include glutamic acid, asparatic acid, lanthionine, β-methyllanthionine, cystathionine, diencolic acid, felinine, aminomalonic acid, β-oxyasparginic acid, α-amino-α-methylsuccinic acid, β-oxyglutamic acid, γ-oxyglutamic acid, γ-methylglutamic acid, γ-methyleneglutamic acid, γ-methyl-γ-oxyglutamic acid, α-aminoadipic acid, α-amino-γ-oxyadipic acid, α-aminopimelic acid, α-amino-γ-oxypimelic acid, β-aminopimelic acid, α-aminosuberic acid, α-aminosebacic acid, and pantotenic acid.

The top layer of human skin and hair is a layer known as the horny layer, and this horny layer consists of cells that have been keratinized (keratinized cells), and “intercellular lipids” that fill in the gaps of the cells. The barrier function of the skin is thought to be primarily dependent on the “intercellular lipids”, and these “intercellular lipids” are primarily made of ceramides, and align in plane to adopt a “lamellar structure” that retains moisture between the structural elements. Degradation of the barrier function of skin and hair is thought to be caused by a loss of ceramides from the horny layer for some reason, and by disorganizing of the lamellar structure of the intercellular lipids.

It is hypothesized that the multi-chain multiple hydrophilic group-type compound expressed by formula (1) can easily align in a plane because of the bulk balance and positional relationship of the hydrophobic and hydrophilic groups, and when the multi-chain multiple hydrophilic group-type compound permeates into the horny layer of skin where the intracellular lipid lamellar structure has become disorganized, the compound can easily create the lamellar structure together with the remaining ceramide. Furthermore, the surface activity and the permeability are high, so the compound easily penetrates into the horny layer when applied to the surface of the skin. Therefore, it is thought that when the multi-chain multiple hydrophilic group-type compound expressed by formula (1) is applied to the surface of the skin, the compound permeates into the horny layer, acts in a manner similar to ceramide replacing the ceramide that was lost, and restores the barrier function of the skin.

The compound expressed by formula (1) is preferably the compound expressed by the following formula (3).

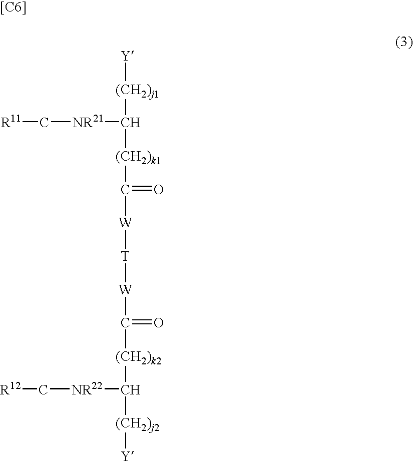

(In formula (3), R¹¹ and R¹² each independently represent a hydrocarbon group which may be substituted with a saturated or unsaturated substitution group of from 8 to 20 carbon atoms, R²¹ and R²² each independently represent either a hydrogen atom or a lower alkyl group of from 1 to 3 carbon atoms that may be substituted with a substitution group, each Y′ independently represents a carboxyl group or a salt thereof, each W independently represents —O—, —S—, or —NR′— (where R′ represents a hydrogen atom or a hydrocarbon group of from 1 to 10 carbon atoms which may be substituted with a substitution group), and T represents a spacer of from 1 to 20 carbon atoms. j¹, j², k¹, and k² independently represent either 0, 1, or 2, but j¹ and k¹, and j² and k² cannot both be 0 at the same time.)

In this case, (A) have three carboxyl groups or salts thereof as hydrophilic groups, and this is preferable because the solubility will be improved and the permeability towards skin and hair will be enhanced as compared to a conventionally known double chain double hydrophilic group-type amphipathic substance or the compound where X has a hydroxyl group as a hydrophilic group.

Specific examples of compounds expressed by formula (1) are shown below.

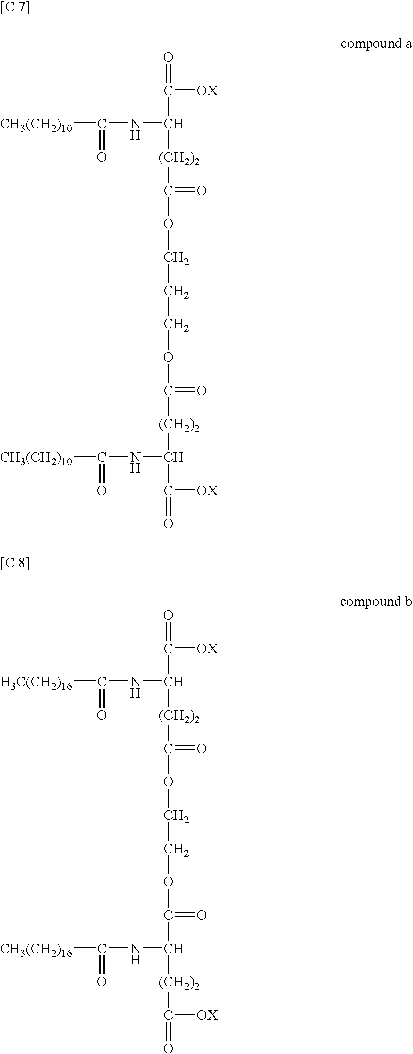

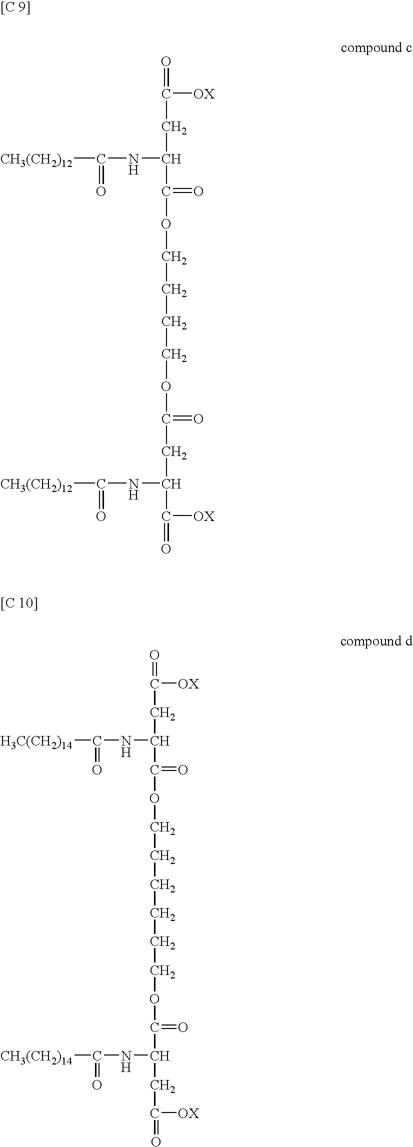

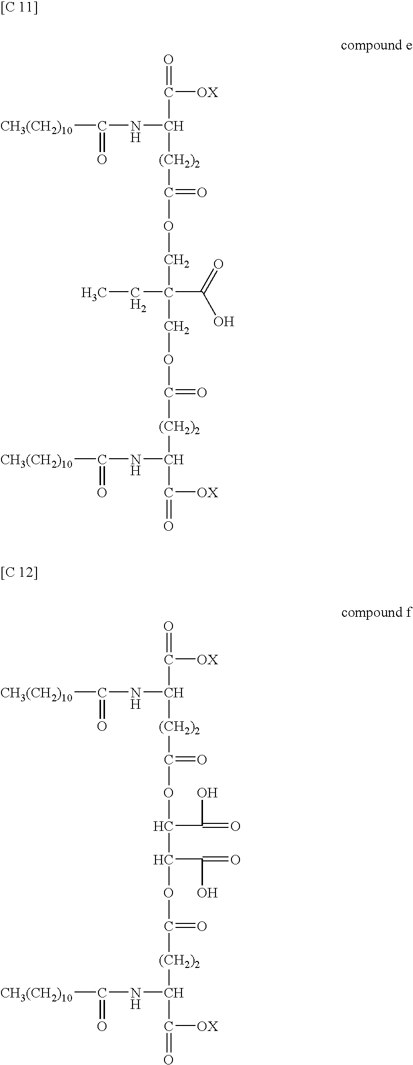

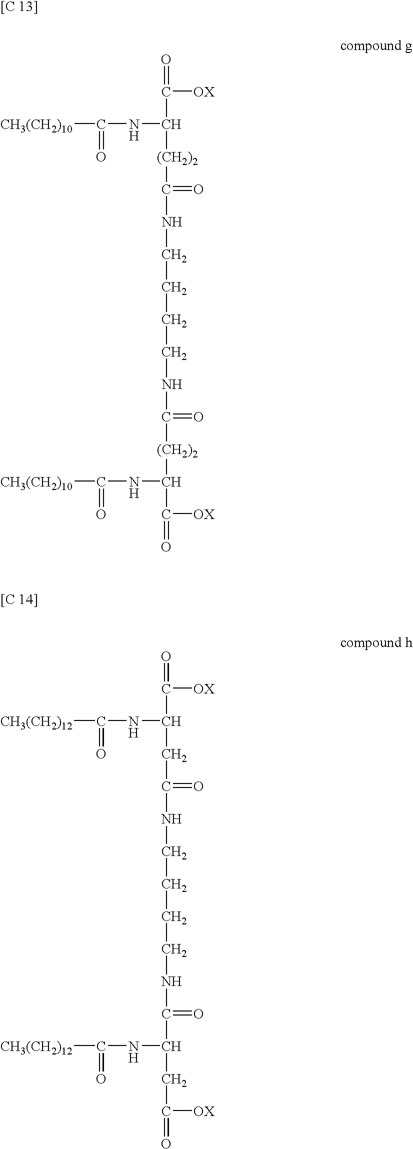

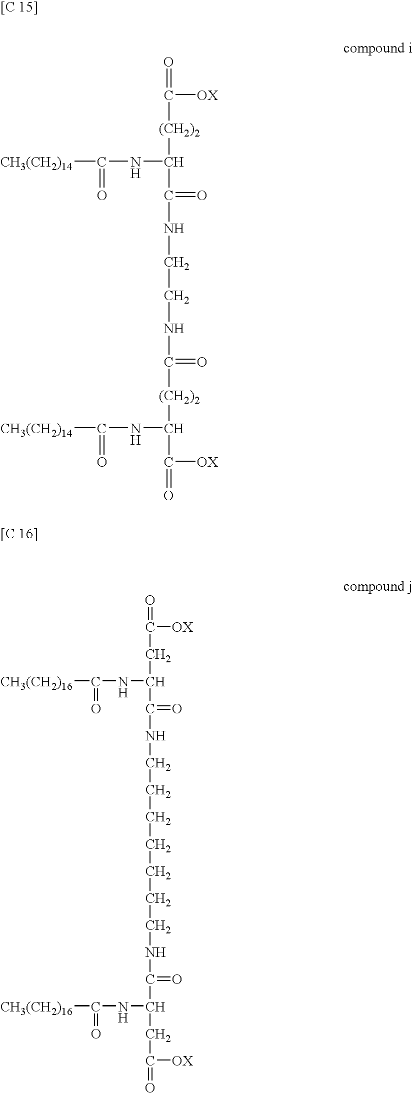

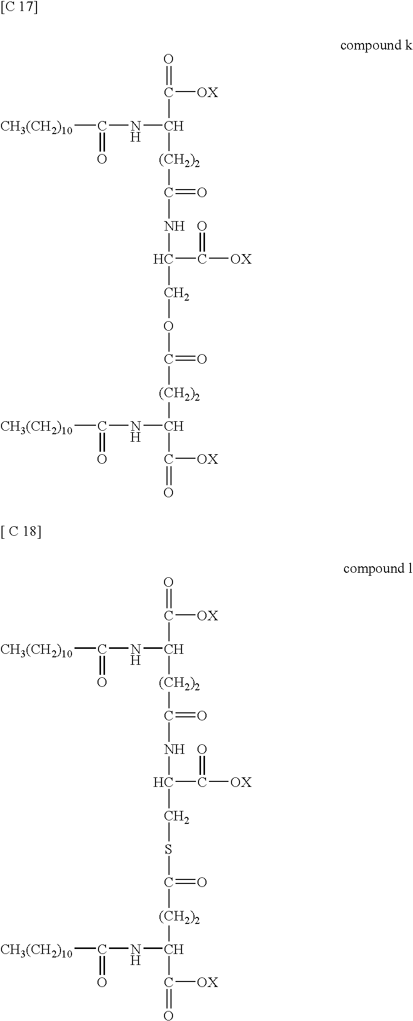

The compound expressed by formula (1) can be prepared by reacting N-acyl acidic amino acid anhydride expressed by formula (4) with a polyhydroxyl compound, polyamino compound, polythiol compound, or a compound which has m functional groups and such as a hydroxyl group, amino group, or thiol group or the like in a molecule, either in water, solvent blends with an organic solvent and water, or an inert solvent such as tetrahydrofuran, benzene, toluene, xylene, carbon tetrachloride, chloroform, and acetone, or without a solvent, at a temperature that is from −5° C. to 200° C. but in any case, above the melting point.

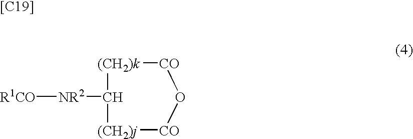

The compound expressed by formula (1) can be prepared for example by dissolving N-acyl acidic amino acid mono lower ester or the like (such as methyl ester or an ethyl ester) and a polyhydroxyl compound, polyamino compound polythiol compound, or a compound having m functional groups such as hydroxyl group, amino group, or thiol group or the like in a molecule, in an appropriate solvent such as dimethylformamide, adding a catalyst such as potassium carbonate, and reacting while heating under reduced pressure at a temperature of −5° C.-250° C., followed by removing the reaction solvent; or by heating and melting them without a solvent, adding a catalyst such as sodium hydroxide and performing an ester replacement reaction at a temperature of room temperature to 250° C.

The formulation ratio of the body surface protecting composition of the present invention is not particularly restricted, but the amount of multi-chain multiple hydrophilic group-type compound of (A) is preferably from 0.001 to 20 mass %, more preferably from 0.01 to 10 mass %, and yet more preferably from 0.01 to 5 mass %. If the amount of multi-chain multiple hydrophilic group-type compound is below the aforementioned range, the effect of improving or preventing a degradation in the barrier function of the skin may not be sufficient.

The oil-based component of (B) that is added in a embodiment of the body surface protecting composition of the present invention can be a volatile or non-volatile oil, including natural ceramide such as ceramide 1, ceramide 2, ceramide 3, ceramide 4, ceramide 5, ceramide 6, ceramide 7, phytoceramide, N-oleoyl sphingosine, N-(12-hydroxyoctadecanoyl) sphingosine, N-(16-hydroxyhexadecanoyl) sphingosine, glycosphingolipid, (N-acyl) phytosphingosine, and animal brain extract; synthetic ceramides such as photoactive ceramide 2 and photoactive ceramide 3; pseudocermides such as O-acyl ceramide, stearoyl dihydrosphingosine, stearoyl phytosphingosine; higher fatty acids such as lower acid, myristic acid, palmitic acid, stearic acid, isostearic acid, oleic acid, behenic acid, undecylenic acid, lanolin fatty acid, hardened lanolin fatty acid, soft lanolin fatty acid, linoleic acid, linolenic acid eicosapentaenoic acid, and 12-hydroxystearic acid; cholesterol esters such as cholesterol palmitate, cholesterol isostearate, di(cholesterol, 2-octyldodecyl) acylglutamate, cholesterol lanolate, and cholesterol hydroxystearate; animal or plant sterols such as cholesterol and phytosterol as well as derivatives thereof; phosphosphingolipid such as sphingomyelin; phospholipids such as lecithin derivatives; fats and oils such as avocado oil, turtle oil, corn oil, almond oil, olive oil, cacao oil, sesame oil, safflower oil, soybean oil, camellia oil, apricot oil, castor oil, grape seed oil, macadamia nut oil, mink oil, cottonseed oil, Japan wax, coconut oil, egg yolk oil, palm oil, palm kernel oil, triisooctanoic acid glycerin, tri-2-ethylhexanoic acid glyceryl cholesterol fatty acid ester, wheat germ oil, camellia kissi seed oil, linseed oil, oenothera tetraptera oil, perilla oil, peanut oil, tea seed oil, kaya oil, rice bran oil, cinnamon bark oil, Japan tung oil, jojoba oil, germ oil, glycerin trioctanoate, glycerin triisopalmitate, horse oil, hardened coconut oil, beef tallow, hoof oil, sheep tallow, hardened beef tallow, pork tallow, beef bone oil, Japan wax kernel oil, hardened oil, Japan wax, and hardened castor oil; hydrocarbons such as liquid paraffin, Vaseline, ceresin, microcrystalline wax, isoparaffin, ozokerite, squalene, pristane, and squalane; waxes such as yellow beeswax, whale oil, lanolin, carnauba wax, candelilla wax, cotton wax, bayberry wax, privet wax, montan wax, bran wax, lanolin, kapok wax, lanolin acetate, liquid paraffin, sugar cane wax, isopropyl lanolate, hexyl laurate, reduced lanolin, jojoba wax, hardened lanolin, shellac wax, polyoxyethylene lanolin alcohol ether, polyoxyethylene lanolin alcohol acetate, polyethylene glycol lanolate, polyoxyethylene hydrogenated lanolin alcohol ether and derivatives thereof; higher alcohols such as lauryl alcohol, cetanol, cetostearyl alcohol, stearyl alcohol, oleyl alcohol, behenyl alcohol, lanolin alcohol, hydrogenated lanolin alcohol, hexyldecanol, and octyldodecanol; ester oils such as isopropyl myristate, butyl stearate, cetyl octanoate, octyldodecyl myristate, isopropyl palmitate, hexyl laurate, myristyl myristate, decyl oleate, hexyldecyl dimethyloctanoate, cetyl lactate, myristyl lactate, lanolin acetate, isocetyl stearate, isocetyl isostearate, cholesterol 12-hydroxystearate, ethylene glycol di-2-ethylhexanoate, dipentaerythritol fatty acid ester, N-alkyl glycol monoisostearate, neopentyl glycol dicapric acid, diisosterile malate, glycerin di-2-heptylundecanoate, trimethylolpropane tri-2-ethylhexanoate, trimethylolpropane triisostearate, pentaerythritol tetra-2-ethylhexanoate, glycerin tri-2-ethylhexanoate, trimethylolpropane triisostearate, cetyl-2-ethyl hexanoate, 2-ethylhexyl palmitate, glycerin trimyristate, glyceride tri-2-heptylundecanoate, methyl castor oil fatty acid ester, oleoyl oleate, cetostearyl alcohol, acetoglyceride, 2-heptylundecyl palmitate, diisopropyl adipate, N-lauroyl-L-glutamic acid-2-octyldodecyl ester, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethylhexyl succinate, ethyl acetate, butyl acetate, amyl acetate, and triethyl citrate; and silicones such as metal soap, dimethyl polysiloxane, polyether modified silicone, alcohol modified silicone, methylphenyl polysiloxane, epoxy modified silicone, fluorine modified silicone, alkyl modified silicone, alkyloxy modified silicone, amino modified silicone, and volatile silicone.

These substances can be used independently, or in combination of two or more. When two or more types are used in combination, a premix of materials blended beforehand can be used.

(B) is preferably a material (liquid crystal formation aid) that has excellent performance in accelerating the liquid crystal formation of (A).

Oil components which have an excellent performance at accelerating the liquid crystal formation of (A) include natural, synthetic, or pseudo-ceramides, cholesterol esters, sterols and derivatives thereof, sphingophospholipids, phospholipids, higher fatty acids, and higher alcohols.

Specific examples include cholesterol esters such as cholesterol palmitate, cholesterol isostearate, di(cholesterol, 2-octyldodecyl) acylglutamate, cholesterol lanolate, and cholesterol hydroxystearate; animal or plant sterols such as cholesterol and phytosterol as well as derivatives thereof; higher fatty acids such as lower acid, myristic acid, palmitic acid, stearic acid, isostearic acid, oleic acid, behenic acid, undecylenic acid, lanolin fatty acid, hardened lanolin fatty acid, soft lanolin fatty acid, linoleic acid, linolenic acid eicosapentaenoic acid, and 12-hydroxystearic acid.

Of these, at least one type of sterol compound selected from cholesterol, cytosterol, lanosterol, phytosterol, dihydrocholesterol, dihydrolanosterol, dehydrosterol and esters thereof and a higher fatty acid or a salt thereof having from 6 to 22 carbon atoms are preferable.

Of these, cholesterol, cytosterol, lanosterol, phytosterol, dihydrocholesterol, dihydrolanosterol, dehydrochloresterol and esters thereof are particularly preferable.

The amount of (B) added can be set within a range that does not hinder the effect of the present invention, and from the viewpoint of the effect of accelerating the liquid crystal formation of (A), the molar ratio with regards to component (A) (amount of (B):amount of (A)) is preferably 1:10 or higher and 10:1 or lower, and more preferably the molar ratio with regards to (A) is 1:5 or higher and 5:1 or lower.

The horny layer intercellular component of (C) added to the example of the body surface protecting composition of the present invention is a water-soluble component other than (B) that exists in the horny layer intercellular space, and examples include NMF (natural moisturizing factors) such as amino acids, lactic acid, urea, and citrates.

The water-soluble moisturizing agent (D) added to the preferable embodiment of the body surface protecting composition of the present invention is a water-soluble component that has moisture retaining capability other than (C) and that essentially does not exist naturally in the horny layer intercellular space, and examples include: polyols such as glycerin, diglycerin, polyglycerin, 1,3-butanediol, propanediol, and polyethylene glycol; alkyl glycines such as N-methylglycine, N,N-dimethylglycine, N,N,N-trimethylglycine, and N-ethylglycine; (poly) saccharides and derivatives thereof such as sorbitol, raffinose, pyrrolidone carboxylates, lactate, hyaluronates, ceramides, trehalose, xylobiose, maltose, sucrose, glucose, and vegetable viscosity modifying polysaccharides; and water-soluble moisture retaining substances such as water-soluble chitin, chitosan, pectin, chondroitin sulfate, and other glycosamino glycanes and salts thereof, glycine, serine, theonine, alanine, asparatic acid, thyrosine, valine, luecine, alginine, glutamine, prolinic acid and other amino acids and salts thereof, aminocarbonyl reaction products and other saccharide amino acid compounds, aloe, horse chestnut and other vegetable extracts solutions, urea, uric acid, ammonia, glucosamine, creatine, DNA, RNA, and other nucleic acid related substances.

These substances can be used independently, or in combination of two or more. If two or more types are used in combination, a premix of materials blended beforehand can be used.

The body surface protecting composition of the present invention can be in a variety of forms depending on the objective, including liquid, solid, gel, paste, cream, slurry, emulsion, suspension, mist, liquid crystal, powder, or aerosol or the like. However, the forms are not limited to these examples.

The body surface protecting composition of the present invention can be unneutralized or neutralized using a basic substance, depending on the application and objective.

The multi-chain multiple hydrophilic group-type compound will have enhanced water solubility if neutralized by a basic substance, so when the body surface protecting composition of the present invention is a water based composition, neutralizing is preferable for the sake of handling.
In this case, the pH of the water based composition is preferably adjusted to from 3 to 12 by adjusting the degree of a neutralization using the basic substance, but a pH from 4.5 to 11 is more preferable, and a pH from 5 to 8 is still more preferable.

The body surface protecting composition of the present invention can be used as an agent to protect the surface of the body, or can be broadly used as a composition that contact with humans such as cosmetics and toiletries or medicines.

In the present invention, a body surface protecting agent refers to a substance that is applied externally to the surface of the body (skin, hair, or the like) and prevents or reduces damage to the surface of the body.

In the present invention, the term cosmetics and toiletries refers to quasi drug and cosmetic products as defined in the Japanese Pharmaceutical Affairs Law, and specific examples include quasi drug such as mouthwash, underarm deodorant, bath dusting powder, hair growth stimulants, hair removal products, hair dyes, permanent wave solutions, bathing salts, medicinal cosmetics, and medicated toothpaste; and cosmetics, such as cleaning cosmetics such as cosmetic soaps, facial washes (as cream, paste, liquid, gel, granule, outer, or aerosol, or the like), shampoo, and rinse; scalp and hair cosmetics such as hair dyes, hair treatments (as a cream, mist, oil, gel, or other form and also including split end coating agents), and hair setting agents (hair grease, setting lotion, curling lotion, pomade, stick, fragrant hair oil, hairspray, hair mist, hair liquid, hair foam, hair gel, and water grease); basic cosmetics, such as general creams, emulsions (cleansing cream, cold cream, vanishing cream, and cream, and the like), shaving cream (after shave cream, shaving cream, and the like), skin lotion (hand lotion, general cosmetic lotion, and the like), cologne, shaving lotion (after shaving lotion, shaving lotion and the like), cosmetic oils, and packs; makeup cosmetics such as skin whiteners (whitening cream, whitening solids, whitening powder, talcum powder, whitening paste, baby powder, body powder, whitening water, and the like), powder, foundation (as cream, liquid, or solid, or the like), rouge, eyebrow pencil, eye cream, eye shadow, and mascara; perfumes such as standard perfume, paste perfume, and powder perfume; gel, liquid, or ceramic type fragrances, air fresheners, and deodorizers; suntan and sunblock cosmetics such as suntan or sunblock creams, suntan or sunblock lotions, and suntan or sunblock oils; nail cosmetics such as nail creams, fingernail polish, and fingernail polish remover; eyeliner cosmetics; mouth and lip cosmetics such as lipstick and lip creams; oral cosmetics such as toothpaste; and bath cosmetics such as bath salt, bath oil, and bubble bath.

Various types of materials that are normally used in external preparations such as cosmetics and toiletries or medicinal products can be added to the body surface protecting composition of the present invention based on the application and objectives, to the degree that the object of the present invention is not hindered.

Specific examples include dispersing agents such as natural gums like arabia gum and tragacanth gum, glucosides such as saponin, cellulose derivatives such as methylcellulose, carboxycellulose, and hydroxymethylcellulose, natural polymers such as lignin sulfonate and shellac, anionic polymers such as polyacrylate, styrene-acrylic acid copolymer salts, vinyl naphthalene-maleic acid copolymer salts, sodium salts of β-naphthalene sulfonate formalin condensates, and phosphates, and nonionic polymers such as polyvinyl alcohol, polyvinylpyrrolidone, and polyethylene glycol; high level fatty acid salt (soap); high-level fatty acids salts with a hydrophobic group containing between 8 and 20 carbons; N-acylamino acid based anionic surfactants (where the acyl group has between 8 and 20 carbons as described above, and the component amino acid is an amino acid such as the aforementioned acidic amino acids like glutamic acid and aspartic acid, as well as glycine, alanine, valine, leucine, isoleucine, proline, methionine, cystine, tryptophan, tyrosine, phenylalanine, asparagine, glutamine, serine, threonine, oxyproline, β-aminopropionic acid, γ-aminobutanoic acid, anthranilic acid, m-aminobenzoic acid, and p-aminobenzoic acid); anionic surfactants such as alkyl ether carboxylates, amide ether carboxylates, alkyl sulfate ester (AS), polyoxyethylene alkyl ether sulfate esters (AES), alkyl ether sulfate, sulfates of high-level fatty acid esters, sulfates of high-level fatty acid alkylolamides, sulfated oils, polyoxyethylenated phenyl ether sulfates, α-olefin sulfonates (AOS), alkylbenzene sulfonates, alkylnaphthalene sulfonates, alkyl sulfonates (SAS), dialkylsulfosuccinate, α-sulfonated fatty acid salts, alkane sulfonates, sulfonates of higher-level fatty acid esters, α-sulfonated fatty acid salts, sulfonates of higher-level fatty acid amides, N-acyl-N-alkyltaurate, N-acyl-N-methyltaurate, alkyl phosphates, alkyl ether phosphates, polyoxyethylene alkyl ether phosphates, polyoxyethylene alkylphenyl ether phosphates, and naphthalenesulfonate formalin condensate; amphoteric surfactants such as alkylbetaines, alkylamidebetaines, alkylsulfobetaines, imidazolinium betaines, and lecithins; oxygenated ethylene condensate type nonionic surfactants such as polyoxyethylene alkyl ether (AE), polyoxyethylene alkylphenyl ether, polyoxyethylene polystearylphenyl ether, polyoxyethylene polyoxypropylene glycol, polyoxyethylene polyoxypropylene alkyl ether, polyoxyalkylene fatty acid ester, polyoxyalkylene sorbitan fatty acid ester, polyoxyalkylene fatty acid alkanolamide, polyoxyalkylene alkyl glycoside, polyoxyalkylene harden castor oil, polyoxyalkylene alkylamine, and polyoxyalkylene alkylphenyl ether; polyhydric alcohol ester type nonionic surfactants such as polyhydric alcohol fatty acid part ester, polyoxyethylene polyhydric alcohol fatty acid part ester, polyoxyethylene fatty acid ester, (poly)glycerin fatty acid ester, polyoxyethylene hardened castor oil, polyoxyethylene alkylamine, triethanolamine fatty acid part ester, alkyl polyglycoside, propylene glycol fatty acid ester, sorbitan fatty acid ester, and sucrose or the like fatty acid esters; nonionic surfactants such as fatty acid alkanolamide, sugar amine acyl compounds, triethanolamine fatty acid part ester, fatty acid alkylolamide, and alkylamine oxide; cationic surfactants such as primary through tertiary fatty acid amine salts, alkylammonium chloride, tetraalkyl ammonium salt, trialkylbenzyl ammonium salt, alkylpyridinium salt, alkylhydroxylethyl imidazolinium salt, dialkylmorpholinium salt, alkylisoquinolium salt, benzetonium salt, and benzalkonium salt; polymer surfactants such as sodium alginate, starch derivatives, and tragacanth gum; natural surfactants such as phospholipids, lecithin, lanolin, cholesterol, and saponin; oil soluble polymers such as hydroxyethyl cellulose, carboxymethyl cellulose, hydroxyethyl cellulose; hydroxypropyltrimethyl ammonium chloride ether, methylcellulose, ethyl cellulose, hydroxypropyl cellulose, methylhydroxypropyl cellulose, soluble starch, carboxymethyl starch, methyl starch, propylene glycol alginate ester, polyvinyl alcohol, polyvinyl pyrrolidone, polyvinyl methyl ether, carboxyvinyl polymer, polyacrylate, guar gum, locust bean gum, queenseed, carrageenan, galactan, arabia gum, pectin, mannan, starch, xanthum gum, dextran, succinoglucane, cardlan, hyaluronic acid, gelatin, casein, albumin, collagen, methoxyethylene maleic anhydride copolymer, amphoteric methacrylate ester copolymer, polydimethylmethylene piperidinium chloride, polyacrylate ester copolymer, polyvinyl acetate, nitrocellulose, and silicone resin; cationic polymers such as cationized cellulose derivative, cationic starch, cationized guar gum derivative, diallylquaternary ammonium salt/acrylamide copolymer, quaternized polyvinyl pyrrolidone derivative, quaternized vinyl pyrrolidone/vinyl imidazole polymer, polyglycol/amine condensate, quaternized collagen polypeptide, polyethyleneimine, cationic silicone polymer, adipic acid/dimethylamino hydroxypropyl diethylene triamine copolymer, polyaminopolyamide, cationic chitin derivative, and quaternized polymer; thickeners and foaming components such as polyethylene glycol fatty acid ester, polyoxyethylene fatty acid ester methylglycoside, and tetradecene sulfonates; sequestering agents such as ethylenediamine tetraacetate and salts thereof, hydroxyethylenediamine triacetate and salts thereof, phosphoric acid, ascorbic acid, succinic acid, gluconic acid, poly phosphates, metaphosphates, and hinokithil (Japanese cypress extract); preservatives and antimicrobial agents such as paraoxybenzoate esters, benzoic acid and salts thereof, phenoxyethanol, hinokithil (Japanese cypress extract), salicylic acid and salts thereof, sorbic acid and salts thereof, dehydroacetic acid and salts thereof, parachlorometacresol, hexachlorophene, boric acid, resorcin, tribromosalan, ortho-phenyl phenol, thiram, photosensitizing dye 201, halocarbane, trichlorocarbanide, tocopherol acetate, zinc pyrithione, phenol, isopropylmethylphenol, 2,4,4-trichloro-2-hydroxyphenol, hexachlorophene, chlorohexidine, benzetonium chloride, benzalkonium chloride, cetylpyridinium chloride, decalinium chloride, stearyldimethyl ammonium chloride, stearyltrimethyl ammonium chloride, cetyltrimethyl ammonium chloride, methylbenzetonium chloride, lauryltrimethyl ammonium chloride, lauroyl colamino formylmethyl pyridinium chloride, trichlosan, and biozole; pH adjusting agents such as citric acid, malic acid, adipic acid, glutamic acid, and asparaginic acid; as well as other dandruff and itch preventing agents such as trichlorocarbanilide, salicylic acid, zinc pyrithione, and isopropylmethyl phenol; ultraviolet light absorbers such as benzophenone derivatives, paraminobenzoic acid derivatives, paramethoxycinnamic acid derivatives, and salicylic acid derivatives; whitening agents such as ascorbic acid and salts thereof (alkali metal salts and alkali earth metal salts such as sodium salt, potassium salt, magnesium salts, and calcium salts, as well as ammonium salts and amino acid salts, and the like), ascorbic acid derivatives (alkyl L-ascorbate ester, L-ascorbic acid phosphate ester and salts thereof, L-ascorbic acid-2-sulfate ester and salts thereof, and L-ascorbic acid glucoside and the like), alkoxysalicylic acid and salts thereof (examples of the alkoxy groups include methoxy groups, ethoxy groups, propoxy groups, an isopropoxy groups, butoxy groups, and isobutoxy groups, and the like), hydroquinone glycoside and derivatives thereof (albutin and the like), kojic acid and derivatives thereof, ellagic acid, chamomile extract, althea extract, liver extract, mulberry bark extract, raspberry extract, apple flavonoid, bran extract, vitamin E and derivatives thereof, hinokithiol, placenta extract, leucinol, camomilla ET, glutathione, clove extract, tea extract, astaxanthin, bovine placenta extract, tranexamic acid and derivatives thereof, resorts and derivatives thereof, azulene, and γ-hydroxybutanoic acid; blood circulation enhancers such as swertia extract, cepharanthine, vitamin E and derivatives thereof, and γ-oryzanol; local stimulants such as capsicum tincture, ginger tincture, cantharide tincture, and benzyl nicotinate ester; nutrients such as various vitamins and amino acids; female hormones; hair root activator; antiinflammatories such as glycyrrhetic acid, glycyrrhizinic acid derivative, allantoin, azulene, aminocaproic acid, and hydrocortisone; astringents such as zinc oxide, zinc sulfate, allantoin hydroxy aluminum, aluminum chloride, zinc sulfocarbolate, tannic acid, citric acid, and lactic acid; cooling agents such as menthol and camphor; antihistamines such as diphenhydramine hydrochloride, chlorpheniramine maleate, and glycyrrhizinic acid derivatives; silicone based substances such as polymer silicone and cyclic silicone; antioxidants such as tocopherols, BHA, BHT, gallic acid, and NDGA; sebum inhibitors such as estradiol, estrone, and ethinylestradiol; keratin peelers and dissolvers such as sulfur, salicylic acid, and resorcin; α-hydroxyacids such as glycolic acid, lactic acid, malic acid, tartaric acid, and citric acid; cosmetic colorants such as talc, kaolin, sericite, calcium carbonate, zinc oxide, aluminum oxide, magnesium oxide, zirconium oxide, magnesium carbonate, calcium carbonate, barium carbonate, chromium oxide, chromium hydroxide, tar based colorants, mica, (synthetic) sericite, silicon carbide, boron nitride, titanium dioxide, black titanium oxide, navy blue, red iron oxide, black iron oxide, yellow iron oxide, ultramarine, titanium coated mica, bismuth oxychloride, bengara, body pigments, ultramarine pink, ultramarine violet, chromium hydroxide, mica titanium, chromium oxide, aluminum cobalt oxide, carbon black, silicic anhydride, magnesium silicate, bentonite, (synthetic) mica, zirconium oxide, (meta) magnesium aluminosilicate, calcium aluminosilicate, polyethylene powder, nylon powder, polymethylmethacrylate, polyethylene terephthalate-polymethyl methacrylate layered powder, acrylonitrile-methacrylic acid copolymer powder, vinylidene chloride-methacrylic acid copolymer powder, wool powder, silk powder, crystalline cellulose, N-acyllysine, polymethylsilsesquioxane powder, plant fruit or peel powders, bismuth oxychloride, mica titanium, iron oxide coated mica, iron oxide mica titanium, organic pigment treated mica titanium, aluminum powder, fine powdered titanium oxide, fine powdered zinc oxide, fine powdered titanium oxide coated mica titanium, fine powdered zinc oxide coated mica titanium, barium sulfate coated mica titanium, carmine, β-carotene, chlorophyll, sunset yellow FCF, ponceau SX, eosine YS, tetrabromofluorescein, rhodamine B, quinoline yellow SS, quinoline yellow WS, alizanine cyanine green, quinizarin green, resolvin B, resolvin BCA, parmatone red, helindon pink CN, phthalocyanine blue, β-apo-8-carotinal, capsanthin, rilopine, bixin, crocin, canthaxanthin, shisonin, lafanine, ninocyanine, carsamine, safrole yellow, rutine, quercitine, cocoa colorant, riboflavin, laccaic acid, carminic acid, kermesic acid, alizanine, shikonin, alkannin, nikino chrome, hemoglobin, curcumin, and betanin; as well as purified water, pyracantha extract, n-methyl-l-serine, whey, nicotinamide, diisopropylamine dichloroacetate, mevalonic acid, γ-aminobutanoic acid (including γ-amino-β-hydroxybutanoic acid), althea extract, aloe extract, apricot kernel extract, turmeric extract, oolong tea extract, dried seawater, hydrolyzed wheat powder, hydrolyzed, silk, carrot extract, cucumber extract, gentian extract, yeast extract, rice germ extract, comfrey extract, caryophyllaceae extract, rehmannia root extract, lithospermum root extract, white birch extract, peppermint extract, swertia extract, bisabolol, propolis, luffa cylindrica extract, tilia platyphyllos flower extract, hop extract, horse chestnut extract, sapindus mukurossi peel extract, balm mint leaf extract, eucalyptus leaf extract, saxifraga sarementosa extract, rosemary extract, matricaria chamomilla extract, royal jelly extract, seaweed, rice bran, liver, citrus unshiu peel, angelica acutiloba, crushed peach bell, sphingolipids, guaiazulene, and vitamin c and the like.

Of the aforementioned materials, fatty acid diethanolamide, polyoxyethylene dioleic acid methylglucoside, polyethylene glycol distearic acid, tetradecenesulfonate, myristates, and myristyldimethylamine are useful in cleaning applications from the viewpoint of increasing the viscosity and foaming action, and the anionic and cationic surfactants are very useful from the viewpoint of further decreasing irritation.

### EXAMPLES

The present invention will be described below in further detail using examples. Evaluation of the test samples for both the examples of the present invention and the comparative examples was carried out as shown below.

1. Skin Barrier Function Recovery Ratio

The skin barrier recovery ratio is defined as shown below using the transepidermal water loss (TEWL (g/Hr-m²)), and evaluated based on the following criteria. The TEWL was measured using a Cutometer MPA 580 manufactured by COURAGE+KHAZAKA Electronic GMBH Corp.

### (Definition of Skin Barrier Function Recovery Ratio)

TEWL recovery ratio(%)=[((TEWL_{SDS})−(TEWL over time))/((TEWL_{SDS})−(TEWL₀))]×100

Herein, TEWL₀, TEWL_{SDS}, and TEWL over time are defined as shown below.
TEWL₀: The TEWL was measured on the forearm of a panel of 5 healthy males. TE WL₀ was defined as the initial average value of TEWL for the panel.
TEWL_{SDS}: A 5 mass % concentration of sodium lauryl sulfate (SDS) was applied to the forearm of each panel member in order to induce a condition of reduced skin barrier function and rough dry skin, and then the TEWL value was measured. The average value for the TEWL at this time was defined as TEWL_{SDS}. This SDS process was carried out to the extent that the average value of TEWL changed from 7 g/m² hr for the healthy skin to 14-17 g/m² hr for rough dry skin.
TEWL over time: 2 μL/cm² of test sample at an appropriate concentration was openly applied twice a day to the aforementioned rough skin, and the TEWL was measured over time for four days. The average value for the TEWL at this time was defined as TEWL over time.

### (Skin Barrier Function Recovery Ratio Evaluation Criteria)

Skin barrier function recovery ratio is 60% or higher:

[symbol]

Skin barrier function recovery ratio is 40% or higher and less than 60%: ◯

Skin barrier function recovery ratio is 20% or higher and less than 40%: Δ

Skin barrier function recovery ratio is less than 20%: x

2. Horny Layer Condition

Rough dry skin condition was induced in the same way as the evaluation for the skin barrier function recovery rate on the forearm of 5 healthy males, and then the test sample was openly applied, and the condition of the horny layer was observed four days later using a microscope (trademark, USB Microscope M2-S; manufactured by Scalar Corp.) at a zoom of 50×. The condition of the horny layer that was observed in this manner was evaluated according to the following criteria, and based on the average value of a panel of five persons, and evaluation of ◯ was made if the average value was less than 1 point, Δ if the average value was 1 or higher but less than 2, and x if the average value was 2 or higher.

Horny layer cells, skin ridges, skin grooves are clearly arranged: 0 points

Horny layer cells, skin ridges, skin grooves are arranged: 1 points

Horny layer cells, skin ridges, skin grooves are somewhat irregular: 2 points

Horny layer cells, skin ridges, skin grooves are visible but irregular: 3 points

Horny layer cells, skin ridges, skin grooves are blurred and irregular: 4 points

3. Human Patch Results

0.1 g of test sample was impregnated into wax paper of 1 cm², and plastered in a covered condition on healthy skin on the forearm of a panel of 5 healthy males for 24 hours. After 24 hours, the TEWL was measured and the results of this human patch test were evaluated by the following criteria. A large increase rate in TEWL means that the sample tends to destroy the barrier function and cause rough dry skin.

◯: Average increasing rate of TEWL is below 10%

Δ: Average increasing rate of TEWL is 10% or higher, but less than 30%

x: Average increasing rate of TEWL is 30% or higher

4. Uniformity, Smoothness, and Stylability of Hair

The test sample was thoroughly applied to a bundle of damaged hair that was prepared by the method shown below, which was then blow dried while combing. Evaluation was carried out by a panel of 10 persons from the viewpoint of “uniformity from hair roots to hair tips”, “smoothness of the hair”, and the “stylability of the hair”. The evaluation criteria for “uniformity from hair roots to hair tips”, “smoothness of the hair”, and the “stylability of the hair” was as shown below.

7 or more persons gave an assessment of “good”: ◯

From 4 to 6 persons gave an assessment of “good”: Δ

3 or less persons gave an assessment of “good”: x

### (Method for Preparing Damaged Hair)

A bundle of damaged hair was prepared by repeating 4 cycles of a process consisting of applying a permanent wave treatment, brushing 100 times, bleaching, brushing 100 times to a bundle of healthy hair (15 cm, 1 g).

5. Hair Damage Recovery Ratio

The hair damage recovery rate was defined by the following formula using damage recovered hair prepared by immersion in a test sample for 1 minute and then drying the aforementioned damaged hair.

Hair damage recovery ratio(%)=tensile strength of damage recovered hair/tensile strength of healthy hair×100

Wherein the tensile strength is defined as the breaking strength per unit cross-section area, and the breaking strength and the cross-section area of the hair are measured as shown below.

### (a) Breaking Strength

The breaking strength was determined using a tensilon tensile tester.

Tensile speed . . . 20 mm/min

Test hair length . . . 40 mm

Chart speed . . . 100 mm/min

N number=5

### (b) Cross-Sectional Area of a Hair

After having measured the breaking strength, the hair was cut at a position approximately 20 mm from the tip using a razor, and the area was determined by measuring a microscope image.

6. Liquid Crystal Condition

A test sample was dispersed by heating, and then observed at a magnification of 40× using a cross nicol polarized light microscope (CX31-P manufactured by Olympus Corporation), and the liquid crystal condition was evaluated according to the following criteria.

| | |
|---|---|
| 30 or more maltese cross images can be observed from all view | [symbol] |
| Maltese cross images can be observed from all view, but [symbol] | ∘ |
| conditions not met | |
| Maltese cross image can be observed depends on view | Δ |
| Maltese cross image cannot be observed from any view | x |

[Acyl Compound Manufacturing 1]

9.1 g of L-lysine monohydrochloride (0.05 mol) was blended with 57 g of water. This solution was adjusted to a pH of 10-11 with a 25% aqueous solution of sodium hydroxide, while maintaining the reaction temperature at 5° C., and a reaction was carried out by adding 31.1 g (0.1 mol) of N-lauroyl-L-glutamic anhydride over 2 hours while stirring. After continuous stirring of the solution for 2 hours, tertiary butanol was added until the concentration in the solution was 20 mass %, and then 75% sulfuric acid was added in drops to adjust the pH to 2, and the temperature of the solution was adjusted to 65° C. After the addition of the sulfuric acid was complete, stirring was stopped, the solution was allowed to sit for 20 minutes at 65° C., and then the solution separated into an organic layer and an aqueous layer, and the organic layer was separated. Tertiary butanol and water were added to the separated organic layer, and the temperature was maintained at 65° C. while stirring for 20 minutes. After stopping stirring, the solution was allowed to sit and separate into an organic layer and an aqueous layer. After repeating the same water washing operation on the organic layer obtained, the solvent was removed from the organic layer, and then the solution was neutralized to a pH of 6.7 (25° C.) using sodium hydroxide followed by adjusting to a solid content of 30 mass %, and the solution was dried to obtain the compounds (mixtures) expressed in the following formula (5).

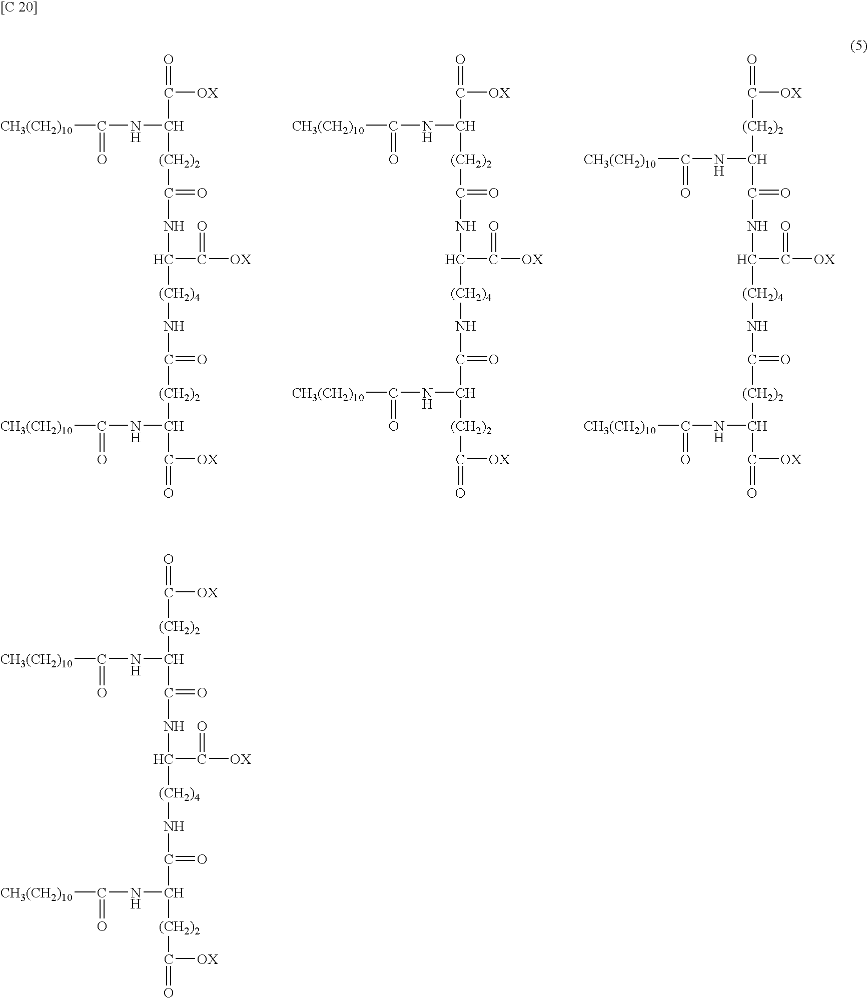

(In formula (5), X independently represents either H or Na.)

[Acyl Compound Manufacturing 2]

The compounds (mixture) as expressed in the following formula (6) were obtained using the same method and conditions as example 1, with the exception that 31.1 g of N-lauroyl-L-glutamic anhydride was replaced with 39.5 g (0.1 mol) of N-stearoyl-L-glutamic anhydride, and instead of neutralizing the pH of the solution to 6.7 (25° C.) and adjusting the solid content to 30 mass %, the pH was neutralized to 7 (60° C.), and the solid content was adjusted to 20 mass %.

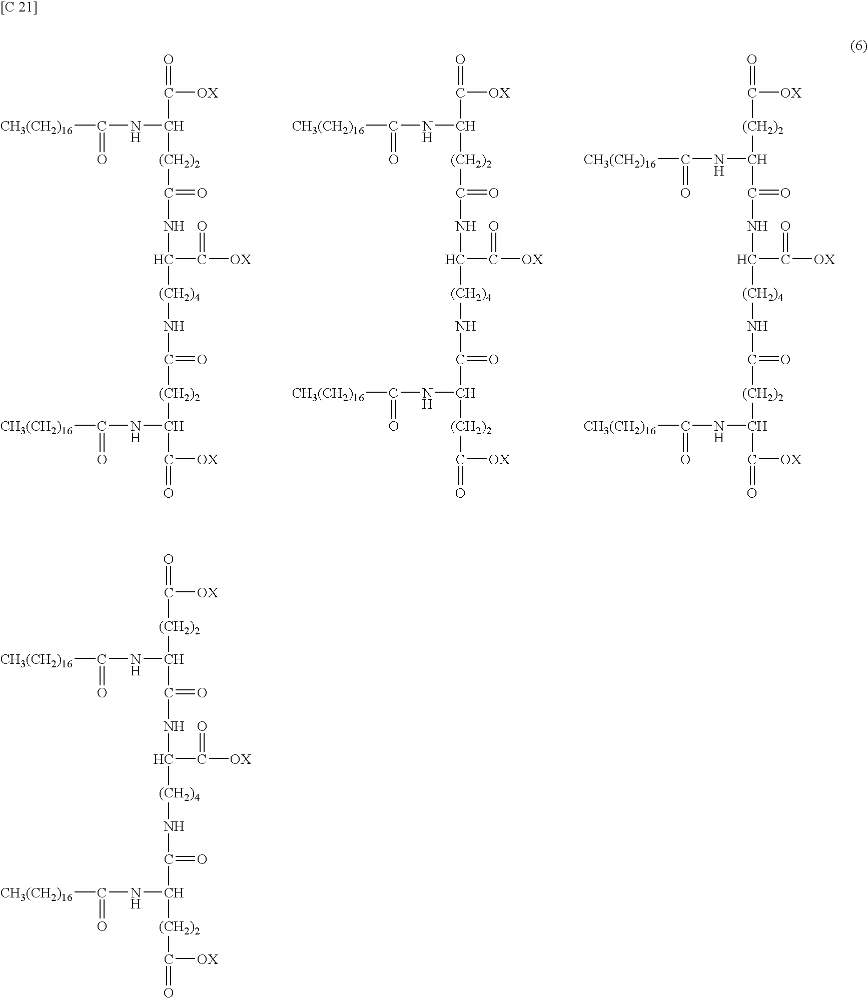

(In formula (6), X independently represents either H or Na.)

[Acyl Compound Manufacturing 3]

The compounds (mixture) as shown in the following formula (7) were obtained using the same method and conditions as example 1, with the exception that the 31.1 g of N-lauroyl-L-glutamic anhydride was replaced with 31.1 g (0.1 mol) of N-cocoyl-L-glutamic anhydride, and potassium hydroxide was used for neutralization when the solution was neutralized and adjusted to a solid content of 30 mass % and a pH of 6.7 (25° C.).

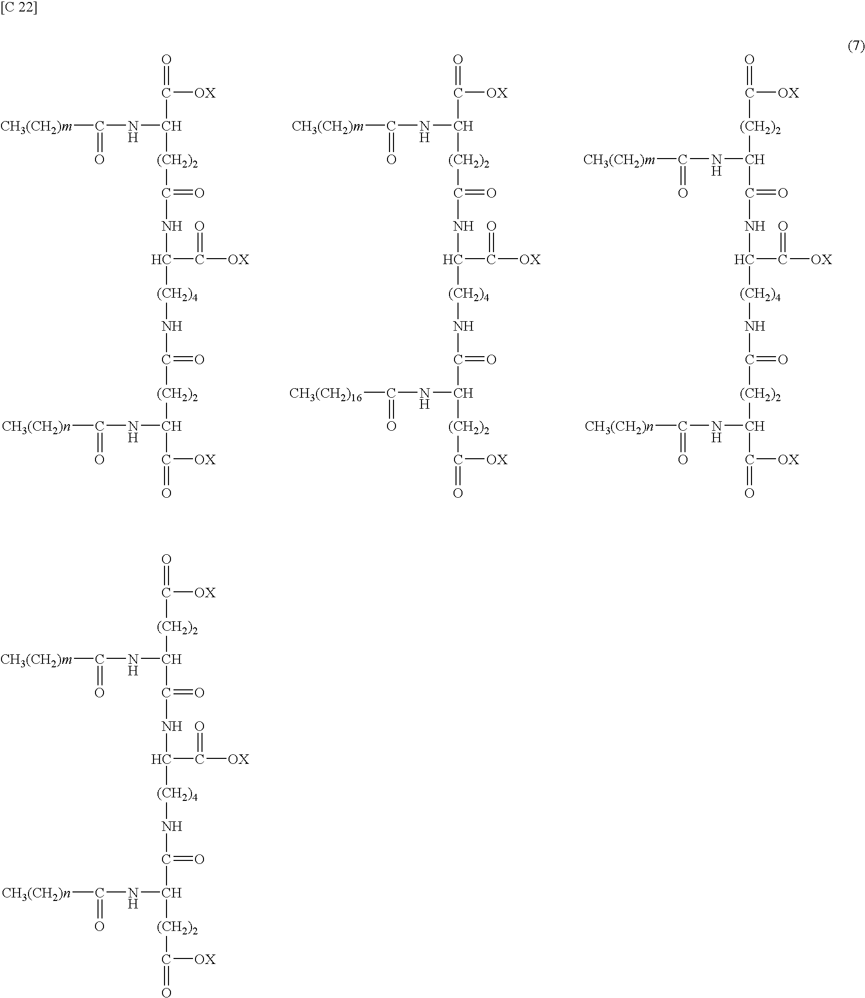

(In formula (7), X independently represents either H or Na, and m and n each independently represent integers from 6 to 16.)

### Examples 1 through 9 and Comparative Examples 1 through 6

The components shown in Table 1 were combined in the amounts (mass parts) shown in Table 1 to prepare body surface protecting compositions for examples 1 through 9 and comparative examples 1 through 6. (Total amount was 100 parts, remainder was purified water). The skin barrier function recovery ratio and the condition of each layer was evaluated for each body surface protecting composition. The results are shown in Table 1.

TABLE 1

| Formulation | Ex 1 | Ex 2 | Exe 3 | Ex 4 | Ex 5 | Ex 6 | Ex 7 | Ex 8 | Ex 9 |
|---|---|---|---|---|---|---|---|---|---|
| Compound of | 5 | — | — | 5 | 1 | — | — | 1 | 2.5 |
| manufacturing 1 | | | | | | | | | |
| Compound of | — | 5 | — | — | — | 1 | — | — | — |
| manufacturing 2 | | | | | | | | | |
| Compound of | — | — | 5 | — | — | — | 1 | — | — |
| manufacturing 3 | | | | | | | | | |
| Sodium | — | — | — | — | — | — | — | 2.5 | — |
| hyaluronate | | | | | | | | | |
| Glycerin | — | — | — | — | — | — | — | — | 2.5 |
| Cholesterol | — | — | — | 0.5 | 0.5 | — | — | — | — |
| Urea | — | — | — | — | — | — | — | — | — |
| Sodium N-lauroyl- | — | — | — | — | — | — | — | — | — |
| L-glutamate | | | | | | | | | |
| L-lysine | — | — | — | — | — | — | — | — | — |
| Purified water | Remainder | Remainder | Remainder | Remainder | Remainder | Remainder | Remainder | Remainder | Remainder |
| Barrier recovery | | | | | | | | | |
| ratio (%) | | | | | | | | | |
| After 1 day | ◯ | ◯ | ◯ | ◯ | ◯ | ◯ | ◯ | ◯ | ◯ |
| After 2 days | [symbol] | [symbol] | [symbol] | [symbol] | ◯ | ◯ | ◯ | ◯ | ◯ |
| After 3 days | [symbol] | [symbol] | [symbol] | [symbol] | [symbol] | ◯ | ◯ | [symbol] | [symbol] |
| After 4 days | [symbol] | [symbol] | [symbol] | [symbol] | [symbol] | [symbol] | [symbol] | [symbol] | [symbol] |
| Condition of horny | [symbol] | [symbol] | [symbol] | [symbol] | ◯ | ◯ | ◯ | ◯ | ◯ |
| layer (after 4 days) | | | | | | | | | |

| | Comp | Comp | Comp | Comp | Com | Comp |
|---|---|---|---|---|---|---|
| Formulation | Ex 1 | Ex 2 | Ex 3 | Ex 4 | Ex 5 | Ex 6 |
| Compound of | — | — | — | — | — | — |
| manufacturing 1 | | | | | | |
| Compound of | — | — | — | — | — | — |
| manufacturing 2 | | | | | | |
| Compound of | — | — | — | — | — | — |
| manufacturing 3 | | | | | | |
| Sodium | — | — | 5 | — | — | — |
| hyaluronate | | | | | | |
| Glycerin | 5 | — | — | — | — | — |
| Cholesterol | — | — | — | — | — | — |
| Urea | — | 5 | — | — | 4 | — |
| Sodium | — | — | — | 5 | — | — |
| N-lauroyl- | | | | | | |
| L-glutamate | | | | | | |
| L-lysine | — | — | — | — | 1 | — |
| Purified water | Remainder | Remainder | Remainder | Remainder | Remainder | Remainder |
| Barrier | | | | | | |
| recovery | | | | | | |
| ratio (%) | | | | | | |
| After 1 day | X | X | X | X | X | X |
| After 2 days | X | X | Δ | Δ | Δ | Δ |
| After 3 days | Δ | Δ | Δ | Δ | Δ | Δ |
| After 4 days | Δ | Δ | Δ | X | X | Δ |
| Condition | Δ | X | Δ | X | X | X |
| of horny layer | | | | | | |
| (after 4 days) | | | | | | |

### Examples 10 through 12 and Comparative Examples 7 through 9

Body surface protecting composition were prepared by combining the components shown in Table 2 in the amount (mass part) shown in Table 2. A human patch test was performed and the effect on the barrier function was confirmed for each composition for protecting human body surface. The results are shown in Table 2.

TABLE 2

| | | | | Comparative | Comparative | Comparative |
|---|---|---|---|---|---|---|
| Formulation | Example 10 | Example 11 | Example 12 | Example 7 | Example 8 | Example 9 |
| Compound of | 5 | — | — | — | — | — |
| manufacturing 1 | | | | | | |
| Compound of | — | 5 | — | — | — | — |
| manufacturing 2 | | | | | | |
| Compound of | — | — | 5 | — | — | — |
| manufacturing 3 | | | | | | |
| Sodium lauroyl | — | — | — | 5 | — | 4 |
| glutamate | | | | | | |
| Potassium cocoyl | — | — | — | — | 5 | — |
| glycine | | | | | | |
| L-lysine | — | — | — | — | — | 1 |
| Purified water | Remainder | Remainder | Remainder | Remainder | Remainder | Remainder |
| Human patch test | | | | | | |
| After 1 day | ◯ | ◯ | ◯ | Δ | X | Δ |
| After 2 days | ◯ | ◯ | ◯ | X | X | X |

*The values in the table are shown as mass %. The total was made to be 100 mass %.

Compositions containing the multi-chain multiple hydrophilic group-type compound of manufacturing examples 1 through 3 were confirmed as not having a negative effect on the barrier function. On the other hand, a composition containing sodium lauroyl glutamate, a composition containing sodium lauroyl glutamate and lysine, and a composition containing potassium cocoyl glycine, which are single chain-type compounds that have basic constitutional units similar to the multi-chain multiple hydrophilic group-type compounds of examples 1 and 3 were confirmed as degrading the barrier function.

From the above, it was confirmed that multi-chain multiple hydrophilic group-type compounds have an effect on improving the barrier function of skin and hair originating from their overall structure.

### Example 13

A cream was prepared as shown below. Stability, skin irritability, and effect of improving the horny layer condition of the cream were all favorable.

| (Component) | (mass parts) |
|---|---|
| (1) Stearic acid | 6 |
| (2) Cetyl alcohol | 4 |
| (3) Butyl stearate | 6 |
| (4) Propylene glycol | 5 |
| (5) Glycerin monostearate | 2 |
| (6) potassium hydroxide | 0.3 |
| (7) Acyl compound of manufacturing 1 | 1 |
| (8) Perfume | as appropriate |
| (10) Preservative and antioxidant | as appropriate |
| (11) Purified water | 75.7 |

### Example 14

A cream was prepared as shown below. Stability, skin irritability, and effect of improving the horny layer condition of the cream were all favorable.

| (Component) | (mass parts) |
|---|---|
| (1) Stearic acid | 2 |
| (2) Stearyl alcohol | 5 |
| (3) Hydrated lanolin | 4 |
| (4) Squalane | 10 |
| (5) Octyldodecanol | 9 |
| (6) 1,3-butylene glycol | 6 |
| (7) polyethylene glycol (mw 1500) | 5 |
| (8) Acyl compound of manufacturing 2 | 0.5 |
| (9) Perfume | as appropriate |
| (10) Preservative and antioxidant | as appropriate |
| (11) Purified water | 53 |

### Example 15

A lotion was prepared as shown below. Stability, skin irritability, and effect of improving the horny layer condition of the lotion were all favorable.

| (Component) | (mass parts) |
|---|---|
| (1) Stearic acid | 2 |
| (2) Cetyl alcohol | 1.5 |
| (3) Vaseline | 4 |
| (4) Squalane | 4 |
| (5) Glycerol tri-2-ethylhexanoate ester | 3 |
| (6) Dipropylene glycol | 5 |
| (7) polyethylene glycol (mw 1500) | 3 |
| (8) Acyl compound of manufacturing 3 | 1 |
| (9) Triethanolamine | 1 |
| (10) Perfume | as appropriate |
| (11) Preservative and antioxidant | as appropriate |
| (12) Purified water | 75.5 |

### Example 16

An emulsion was prepared as shown below. Stability, skin irritability, and effect of improving the horny layer condition of the emulsion were all favorable.

| (Component) | (mass parts) |
|---|---|
| (1) Cetyl alcohol | 1 |
| (2) Beeswax | 0.5 |
| (3) Vaseline | 2 |
| (4) Squalane | 5 |
| (5) Dimethyl polysiloxane | 3 |
| (6) Ethanol | 5 |
| (7) Glycerin | 5 |
| (8) Acyl compound of manufacturing 1 | 1 |
| (9) 1,3-butylene glycol | 3 |
| (10) Carboxyvinyl polymer | 0.2 |
| (11) Perfume | as appropriate |
| (12) Preservative and antioxidant | as appropriate |
| (13) Purified water | 74.3 |

### Example 17

A gel was prepared as shown below. Stability, skin irritability, and effect of improving the horny layer condition of the gel were all favorable.

| (Component) | (mass parts) |
|---|---|
| (1) polyethylene glycol (mw 1500) | 9 |
| (2) Dipropylene glycol | 7 |
| (3) Methyl cellulose | 0.2 |
| (4) Carboxyvinyl polymer | 0.4 |
| (5) Acyl compound of manufacturing 3 | 1 |
| (6) potassium hydroxide | 0.1 |
| (7) Perfume | as appropriate |
| (8) Preservative and antioxidant | as appropriate |
| (9) Chelating agent | as appropriate |
| (10) Purified water | 82.3 |

### Example 18

A facial mask was prepared as shown below. Stability, skin irritability, and effect of improving the horny layer condition of the facial mask were all favorable.

| (Component) | (mass parts) |
|---|---|
| (1) 1,3-butylene glycol | 5 |
| (2) Polyvinyl alcohol | 15 |
| (3) Carboxymethyl cellulose | 5 |
| (4) Ethanol | 12 |
| (5) Acyl compound of manufacturing 1 | 1 |
| (6) Preservative | as appropriate |
| (7) Perfume | as appropriate |
| (8) Purified water | amount to make the |
| | total 100 parts |

### Example 19

A lotion was prepared as shown below. Stability, skin irritability, and effect of improving the horny layer condition of the lotion were all favorable.

| (Component) | (mass parts) |
|---|---|
| (1) Ethanol | 55 |
| (2) Dipropylene glycol | 2.5 |
| (3) Acyl compound of manufacturing 2 | 1 |
| (4) Antimicrobial agent | as appropriate |
| (5) Perfume | as appropriate |
| (6) Purified water | to make 100 parts |

### Examples 20 through 24, Comparative Examples 10, 11

### Preparation of Body Surface Protecting Composition for Example 20 and Comparative Example 10

The body surface protecting composition for example 20 was prepared by combining the components in the amount (mass part) shown in Table 3. Comparative Example 10 contained only purified water.

### Preparation of Body Surface Protecting Composition for Examples 21 Through 23 and Comparative Example 11

The body surface protecting compositions for Example 21 through 23 were prepared by combining the components shown in Table 3 and the following Base Emulsion A in the amount (mass part) shown in Table 3. Base Emulsion A was used alone as the Comparative Example 11.

### (Base Emulsion A)

| | |
|---|---|
| Liquid paraffin | 10 mass parts |
| SEF-320A (product of Nihon Emulsion Co. Ltd.) | 3 mass parts |
| 1,3-butylene glycol | 5 mass parts |
| Cetyl alcohol | 2 mass parts |
| Water | remainder |

### Preparation of Body Surface Protecting Composition for Example 24

The body surface protecting composition for Example 24 was prepared by combining the components shown in Table 3 and the following Base Emulsion B in the amount (mass part) shown in Table 3.

### (Base Emulsion B)

| | |
|---|---|
| Squalane | 10 mass parts |
| 1,3-butylene glycol | 5 mass parts |
| Cetyl alcohol | 2 mass parts |
| Water | remainder |

(Evaluation)

The skin barrier function recovery ratio, hair uniformity, smoothness, and stylability, and liquid crystal condition were evaluated for Examples 20 through 24 and Comparative Examples 10 and 11. The results are shown in Table 3.

TABLE 3

| | Example | Example | Example | Example | Example | Comparative | Comparative |
|---|---|---|---|---|---|---|---|
| Formulation | 20 | 21 | 22 | 23 | 24 | Example 10 | Example 11 |
| Compound of manufacturing 1 | 0.5 | — | 0.5 | 0.5 | 1 | — | — |
| Compound of manufacturing 2 | — | 0.5 | — | — | — | — | — |
| Lauric acid | — | — | 0.2 | — | 0.3 | — | — |
| Cholesterol | — | 0.3 | 0.3 | — | 0.5 | — | — |
| Purified water | Remainder | — | — | — | — | Remainder | — |
| Base Emulsion | — | Remainder | Remainder | Remainder | Remainder | — | Remainder |
| Barrier recovery ratio (%) | | | | | | | |
| After 3 days | X | Δ | ◯ | X | ◯ | X | X |
| After 7 days | Δ | [symbol] | [symbol] | ◯ | [symbol] | X | X |
| After 10 days | ◯ | [symbol] | [symbol] | [symbol] | [symbol] | Δ | ◯ |
| After 14 days | [symbol] | [symbol] | [symbol] | [symbol] | [symbol] | ◯ | ◯ |
| Uniformity of hair | ◯ | ◯ | ◯ | ◯ | ◯ | X | X |
| Smoothness of hair | ◯ | ◯ | ◯ | ◯ | ◯ | Δ | Δ |
| Stylability of hair | ◯ | ◯ | ◯ | ◯ | ◯ | Δ | Δ |
| Liquid crystal condition observation results | ◯ | [symbol] | [symbol] | — | [symbol] | X | X |
| Overall evaluation | ◯ | [symbol] | [symbol] | [symbol] to ◯ | [symbol] | X | X |

*The values in the table are shown as mass %. The total was made to be 100 mass %.

Formation of liquid crystal was confirmed in the compositions of Examples 20 through 22 and 24, and in particular, formation of spherical crystals were clearly confirmed in the compositions of Examples 21, 22, and 24.

From the above, it was confirmed that a body surface protecting composition containing multi-chain multiple hydrophilic group-type compound (A) of the present invention formed liquid crystals, and it was also confirmed that the formation of liquid crystal was facilitated when the composition also contained oil-based component (B). Furthermore, when liquid crystals were formed, in addition to the function of accelerating skin barrier recovery, the effect of improving hair, particularly hair tips, that had been damaged was also confirmed.

### Example 25

Body surface protecting compositions were prepared by combining the components shown in Table 4 in the amount (mass part) shown in Table 4. The liquid crystal condition of these compositions was evaluated. The results are shown in Table 4.

TABLE 4

| Compound of | Choles- | Lauric | Base | Liquid crystal |
|---|---|---|---|---|
| manufacturing 1 | terol | acid | Emulsion (A) | formation |
| 0.5 | 16 | — | Remainder | x |
| 0.5 | 12 | — | Remainder | Δ |
| 0.5 | 4.6 | — | Remainder | Δ |
| 0.5 | 2.3 | — | Remainder | ∘ |
| 0.5 | 1.7 | — | Remainder | ∘ |
| 0.5 | 1.2 | — | Remainder | [symbol] |
| 0.5 | 0.23 | — | Remainder | [symbol] |
| 0.5 | 0.046 | — | Remainder | [symbol] |
| 0.5 | 0.031 | — | Remainder | ∘ |
| 0.5 | 0.023 | — | Remainder | ∘ |
| 0.5 | 0.012 | — | Remainder | Δ |
| 0.5 | 0.005 | — | Remainder | Δ |
| 0.5 | 0.003 | — | Remainder | x |
| 0.5 | — | 8.4 | Remainder | x |
| 0.5 | — | 6 | Remainder | Δ |
| 0.5 | — | 2.4 | Remainder | Δ |
| 0.5 | — | 1.2 | Remainder | ∘ |
| 0.5 | — | 0.90 | Remainder | ∘ |
| 0.5 | — | 0.60 | Remainder | [symbol] |
| 0.5 | — | 0.12 | Remainder | [symbol] |
| 0.5 | — | 0.024 | Remainder | [symbol] |
| 0.5 | — | 0.016 | Remainder | ∘ |
| 0.5 | — | 0.012 | Remainder | ∘ |
| 0.5 | — | 0.0060 | Remainder | Δ |
| 0.5 | — | 0.0024 | Remainder | Δ |
| 0.5 | — | 0.0017 | Remainder | x |

* The figures in the table are shown in mass %. The total was made to be 100 mass %.

Observations were made at 40× zoom using a cross nicol polarized light microscope. [symbol]
: 30 or more maltese cross images can be observed from all view
◯: Maltese cross images can be observed from all view
Δ: Maltese cross image can be observed depends on view
x: Maltese cross image can not be observed from any view

Formation of liquid crystals was confirmed in the composition where cholesterol (molecular weight 387) and lauric acid (molecular weight 200) were added with a molar ratio to a multi-chain multiple hydrophilic group-type compound (molecular weight 835) of from 1/50 to 50/1, and in particular, formation was clearly confirmed in the composition with a molar ratio of from 1/10 to 10/1, and very remarkable formation of liquid crystals was confirmed in the composition with a molar ratio of from 1/5 to 5/1.

From the above, it was confirmed that a composition containing multi-chain multiple hydrophilic group-type compound (A) of the present invention could easily form a liquid crystal composition if oil-based component (B) was used in combination in a specific range within the ratio.

### Examples 26 Through 28, Comparative Examples 12

Body surface protecting compositions were prepared by combining the components shown in Table 5 in the amount (mass part) shown in Table 5. Comparative Example 12 was purified water alone. The hair damage recovery rate was measured for each composition. The results are shown in Table 5.

TABLE 5

| | Example | Example | Example | Comparative | Untreated |
|---|---|---|---|---|---|
| Formulation | 26 | 27 | 28 | Example 12 | damaged hair |
| Compound of | 0.01 | 0.01 | 0.01 | — | — |
| manufacturing 1 | | | | | |
| Laurie Acid | — | — | 0.0008 | — | — |
| Cholesterol | — | 0.0015 | — | — | — |
| Purified water | Remainder | Remainder | Remainder | Remainder | — |
| Damage | 94% | 100% | 99% | 83% | 81% |
| recovery ratio | | | | | |

*The figures in the table are shown in mass %. The total was made to be 100 mass %.

From the above, it was confirmed that a composition containing a multi-chain multiple hydrophilic group-type compound (A) according to the present invention will have an excellent effect of improving hair that has been damaged, and that effect will be increased if an oil-based component (B) is used in combination.

### INDUSTRIAL APPLICABILITY

The body surface protecting composition of the present invention will accelerate recovery of the barrier function of skin and hair when that function has deteriorated due to skin trouble or the like and will prevent degradation of the barrier function, and therefore can be used by adding to cosmetic compositions, pharmaceutical compositions, and body surface protecting agents and the like.

1. (canceled)

2. A body surface protecting composition comprising, (A) at least one type of compound that is a multi-chain multiple hydrophilic group-type compound having two or more hydrophobic groups and two of more hydrophilic groups in the molecule, wherein at least one of the hydrophobic groups is an acyl group, and at least one of the hydrophilic groups is a carboxyl group, a sulfonate group, a sulfate ester group, a phosphate ester group, or a salt thereof.

3. The body surface protecting composition according to claim 2, wherein at least one type of said multi-chain multiple hydrophilic group-type compound is a compound having an amino acid residue in the molecule.

4. The body surface protecting composition according to claim 2, wherein at least one type of said multi-chain multiple hydrophilic group-type compound is a compound expressed by the following formula (1)
[C23]
Qn-X (1)
(In formula (1), X represents a spacer with a molecular weight of one million or less, has m functional group residues that may be the same or different, and may have other functional groups. Q represents a functional group expressed by the following formula (2), and the functional groups may be the same or different. 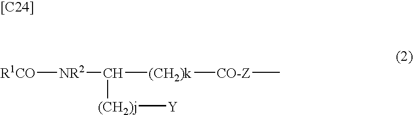
In formula (2), Z represents a site of a bonding with a functional group residue contained in X, R¹ represents a hydrocarbon group that may be substituted with a saturated or unsaturated substitution group of from 2 to 20 carbon atoms, R² represents a hydrogen atom or a lower alkyl group of from 1 to 3 carbon atoms which may also be substituted with a substitution group, and Y represents a carboxyl group, a sulfonate group, a sulfate ester group, a phosphate ester group, or a salt thereof. j and k independently represent either 0, 1, or 2, but j and k cannot both be 0 at the same time. n is an integer from 2 to 20. Furthermore, m is an integer such that m≧n.).

5. The body surface protecting composition according to claim 4, wherein at least one type of said multi-chain multiple hydrophilic group-type compound is a compound expressed by said formula (1) having 2 hydrophobic groups and 3 hydrophilic groups in the molecule, and the number of carbon atoms in X shown in said formula (1) is from 1 to 40.

6. The body surface protecting composition according to claim 4, wherein at least one type of said multi-chain multiple hydrophilic group compound is a compound expressed by the following formula (3) 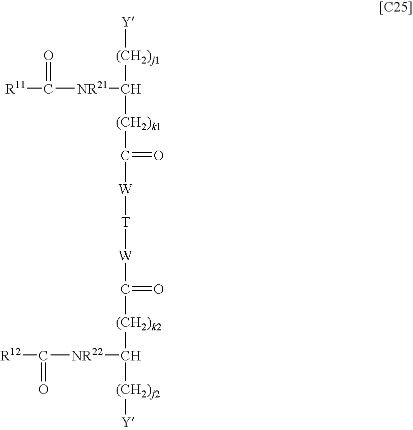
(In formula (3), R¹¹ and R¹² each independently represent a hydrocarbon group which may be substituted with a saturated or unsaturated substitution group of from 8 to 20 carbon atoms, R²¹ and R²² each independently represent either a hydrogen atom or a lower alkyl group of from 1 to 3 carbon atoms that may be substituted with a substitution group, each Y′ independently represents a carboxyl group or a salt thereof, each W independently represents —O—, —S—, or —NR′— (where R′ represents a hydrogen atom or a hydrocarbon group of from 1 to 10 carbon atoms which may be substituted with a substitution group), and T represents a spacer of from 1 to 20 carbon atoms. j¹, j², k¹, and k² independently represent either 0, 1, or 2, but j¹ and k¹, and j² and k² cannot both be 0 at the same time.).

7. The body surface protecting composition according to claim 2, which has an excellent effect of accelerating the recovery of skin barrier function.

8. The composition according to claim 2, wherein said (A) forms liquid crystals.

9. The body surface protecting composition according to claim 2, further comprising, (B) at least one type of oil-based component.

10. The body surface protecting composition according to claim 9, wherein said oil-based component is a liquid crystal forming aid.

11. The body surface protecting composition according to claim 10, wherein said liquid crystal forming aid comprises at least one type of sterol compound.

12. The body surface protecting composition according to claim 11, wherein said sterol compound is at least one selected from a group consisting of cholesterol, cytosterol, lanosterol, phytosterol, dihydrocholesterol, dihydrolanosterol, and dehydrocholesterol and esters thereof.

13. The body surface protecting composition according to claim 10, wherein said liquid crystal forming aid is at least one selected from a group consisting of fatty acids and salts thereof.

14. The body surface protecting composition according to claim 13, wherein said fatty acid is a fatty acid of 6 or more and 22 or less carbon atoms.

15. The body surface protecting composition according to claim 13, wherein said fatty acid is a fatty acid of 8 or more and 16 or less carbon atoms.

16. The body surface protecting composition according to claim 10, wherein the molar ratio of said liquid crystal forming aid to (A) (amount of liquid crystal forming aid: amount of (A)) is 1:10 or higher and 10:1 or lower.

17. The body surface protecting composition according to claim 10, wherein the molar ratio of said liquid crystal forming aid to (A) (amount of liquid crystal forming aid: amount of (A)) is 1:5 or higher and 5:1 or lower.

18. The body surface protecting composition according to claim 2, further comprising (C) a horny layer intercellular component and/or (D) a water soluble moisturizing agent.

19. An external preparation for reducing or preventing allergenic or inflammatory skin disease, comprising the body surface protecting composition according to claim 2.

20. A cosmetic toiletry product comprising the body surface protecting composition according to claim 2.

21. A pharmaceutical product comprising the body surface protecting composition according to claim 2.

22. A body surface protecting agent comprising the body surface protecting composition according to claim 2.

23. A use of the body surface protecting composition according to claim 2 as an external use preparation for reducing or preventing allergenic or inflammatory skin disease.

24. A use of the body surface protecting composition according to claim 2 as a cosmetic toiletry product.

25. A use of the body surface protecting composition according to claim 2 as a pharmaceutical product.

26. A use of the body surface protecting composition according to claim 2 as a body surface protecting agent.

27. A use of the body surface protecting composition according to claim 2 for manufacturing an external preparation for reducing or preventing allergenic or inflammatory skin disease.

28. A use of the body surface protecting composition according to claim 2 for manufacturing a cosmetic toiletry product.

29. A use of the body surface protecting composition according to claim 2 for manufacturing a pharmaceutical product.

30. A use of the body surface protecting composition according to claim 2 for manufacturing a body surface protecting agent.
